(19) United States
(12) Patent Application Publication
(10) Pub. No.: US 2024/0325148 A1
(43) Pub. Date: Oct. 3, 2024

(54) DELIVERY APPARATUS AND METHODS FOR IMPLANTING PROSTHETIC HEART VALVES

(71) Applicant: EDWARDS LIFESCIENCES CORPORATION, Irvine, CA (US)

(72) Inventors: Elazar Levi Schwarcz, Netanya (IL); Eitan Atias, Netanya (IL)

(21) Appl. No.: 18/742,002

(22) Filed: Jun. 13, 2024

Related U.S. Application Data
(63) Continuation of application No. PCT/US2022/053224, filed on Dec. 16, 2022.
(60) Provisional application No. 63/292,285, filed on Dec. 21, 2021.

(51) Int. Cl. A61F 2/24

(52) U.S. Cl. CPC A61F 2/2436

(57) ABSTRACT

A delivery apparatus for a prosthetic heart valve includes a handle with at least one rotatable knob. A pull body is disposed within the cavity and operatively coupled to the knob such that rotation of the knob results in movement of the pull body within the cavity and along a longitudinal axis of the handle. An actuation assembly that can releasably engage an actuator of the prosthetic heart valve includes a sleeve member. An end portion of the sleeve member is disposed within the cavity of the handle and coupled to the pull body such that movement of the pull body by rotation of the knob axially displaces the sleeve member relative to the handle. The sleeve member can be axially displaced to release the actuation assembly from engagement with an actuator of the prosthetic heart valve.

### CROSS-REFERENCE TO RELATED APPLICATIONS

This application is a continuation of International Patent Application No. PCT/US2022/053224, filed Dec. 16, 2022, which claims the benefit of U.S. Provisional Patent Application No. 63/292,285, filed on Dec. 21, 2021, both of which are incorporated by reference herein.

### FIELD

The field relates to implantable prosthetic devices, such as prosthetic heart valves, and to delivery apparatus and methods for implanting prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable.

In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves that rely on a mechanical actuator for expansion can be referred to as “mechanically expandable” prosthetic heart valves. Mechanically expandable prosthetic heart valves can provide one or more advantages over self-expandable and balloon-expandable prosthetic heart valves. For example, mechanically expandable prosthetic heart valves can be expanded to various diameters. Mechanically expandable prosthetic heart valves can also be compressed after an initial expansion (e.g., for repositioning and/or retrieval). However, some known devices and methods can cause rotation or movement of the prosthetic valve during expansion.

Despite the recent advancements in percutaneous valve technology, there remains a need for improved transcatheter heart valves and delivery devices for such valves.

### SUMMARY

Described herein are delivery apparatus and methods for implanting prosthetic heart valves. The disclosed delivery apparatus and methods can, for example, reduce the difficulty and/or the time needed to implant a prosthetic heart valve. The disclosed delivery apparatus are relatively simple and easy to use and include various safeguards, which can help to ensure that the prosthetic heart valve is safely and securely implanted.

A delivery apparatus for a prosthetic implant, such as a prosthetic heart valve, can comprise a handle and one or more shafts coupled to the handle.

In some examples, a delivery apparatus for a prosthetic heart valve can comprise a handle body, a pull body, an actuation assembly, and a knob. The handle has a cavity and defines a longitudinal axis. The pull body is disposed within the cavity and movable within the cavity and along the longitudinal axis. The actuation assembly comprises a sleeve member having an end portion disposed within the cavity and coupled to the pull body such that movement of the pull body along the longitudinal axis axially displaces the sleeve member relative to the handle body. The knob is rotatably mounted on the handle body and operatively coupled to the pull body such that rotation of the knob relative to the handle body moves the pull body within the cavity and along the longitudinal axis.

In some examples, a delivery apparatus for a prosthetic heart valve can comprise a handle body, an actuator driver, a sleeve member, and a pull body. The handle body has a first end, a second end, a cavity between the first end and the second end, and a longitudinal axis extending through the first end, the cavity, and the second end. The actuator driver has a first driver end portion disposed within the cavity, a second driver end portion disposed outside the cavity, and a first axial axis extending through the first driver end portion and the second driver end portion. The sleeve member is operatively coupled to the actuator driver. The sleeve member has a first sleeve end portion disposed within the cavity, a second sleeve end portion disposed outside the cavity, and a second axial axis extending through the first sleeve end portion and the second sleeve end portion, the second axial axis parallel to the first axial axis. The pull body is disposed within the cavity and coupled to the first sleeve end portion. The pull body is movable along the longitudinal axis such that movement of the pull body axially displaces the sleeve member relative to the handle body.

In some examples, a delivery apparatus comprises one or more of the components recited in Examples 1-40 herein.

An assembly can comprise a prosthetic heart valve and a delivery apparatus.

In some examples, a delivery assembly can comprise a prosthetic heart valve, a handle, an actuator driver, a sleeve member, a pull body, and a knob. The prosthetic heart valve comprises a frame and at least one actuator coupled to the frame and operable to move the frame between a radially expanded configuration and a radially compressed configuration. The handle comprises a handle body having a cavity and defining a longitudinal axis. The actuator driver is configured to releasably engage the at least one actuator of the prosthetic heart valve. The sleeve member is operatively coupled to the actuator driver and movable between a first position in which the actuator driver is retained in engagement with the at least one actuator and a second position in which the actuator driver is released from engagement from the at least one actuator. The pull body is disposed within the cavity and coupled to an end portion of the sleeve member, the pull body movable along the longitudinal axis such that movement of the pull body axially displaces the sleeve member between the first position and the second position. The knob is rotatably mounted on the handle body and operatively coupled to the pull body such that rotation of the knob relative to the handle body moves the pull body along the longitudinal axis.

In some examples, a delivery assembly comprises one or more of the components recited in Examples 41-48 herein.

A method of implanting can comprise coupling a prosthetic heart valve to a delivery apparatus, delivering the prosthetic heart valve to an implantation location with the delivery apparatus, and expanding the prosthetic heart valve to a functional size or working diameter using the delivery apparatus.

In some examples, a method of implanting a prosthetic heart valve can comprise engaging an end portion of an actuator driver coupled to a handle with an end portion of an actuator coupled to a frame of the prosthetic heart valve, moving a pull body along a longitudinal axis of the handle in a first direction to extend an end portion of a sleeve member coupled to the pull body over the engaged end portions of the actuator driver and actuator, delivering the prosthetic heart valve to an implantation location, radially expanding the prosthetic heart valve to a functional size, moving the pull body along the longitudinal axis of the handle in a second direction that is opposite to the first direction to retract the end portion of the sleeve member from the engaged end portions of the actuator driver and actuator, and releasing the end portion of the actuator driver from the end portion of the actuator.

In some examples, a method comprises one or more of the operations recited in Examples 49-50 herein.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a prosthetic heart valve.

FIG. 2A is a perspective view of the prosthetic heart valve in a radially expanded configuration with the valvular structure removed, depicting actuator heads at an outflow end of the frame.

FIG. 2B is a perspective view of the prosthetic heart valve in a radially expanded configuration, depicting actuator heads at an inflow end of the frame.

FIG. 3 is a detail view of an actuator of the prosthetic heart valve.

FIG. 4A is a side view of a proximal end portion of a delivery apparatus.

FIG. 4B is a side view of a distal end portion of a delivery apparatus with the prosthetic heart valve in a radially expanded configuration coupled thereto.

FIG. 5 is a cross-sectional view of a shaft assembly of the delivery apparatus, taken along line 5-5 of FIG. 4B.

FIG. 6 is a perspective view of a portion of an actuation assembly of the delivery apparatus.

FIG. 7A is a perspective view of the actuation assembly of the delivery apparatus aligned with an actuator of the prosthetic heart valve.

FIG. 7B is a perspective view of the actuation assembly engaged with the actuator.

FIG. 7C is a perspective view of the outer sleeve of the actuation assembly engaged with the frame of the prosthetic heart valve.

FIG. 8 is a cross-section of a handle of the delivery apparatus, taken along line 8-8 of FIG. 4A.

FIG. 9A is a portion of the handle of the delivery apparatus depicting a gearbox within the handle coupled to a knob of the handle.

FIG. 9B is a perspective view of the gearbox with the gearbox housing depicted as transparent.

FIG. 9C is a perspective view of a portion of the handle of the delivery apparatus depicting compartments inside the gearbox housing.

FIG. 10A is a perspective view of a gear train of the gearbox.

FIG. 10B is a plan view of the gear train in a direction parallel to the longitudinal axis of the handle.

FIG. 10C is a plan view of the gear train in a direction transverse to the longitudinal axis of the handle.

FIG. 11 is another perspective view of the prosthetic heart valve without the valvular structure and illustrating division of the actuation rods into two sets.

FIG. 12 is a schematic of a delivery assembly including the prosthetic heart valve in a radially expanded configuration and the delivery apparatus,

FIG. 13A is a perspective view of a torque limiter for an actuator driver.

FIG. 13B is a cross-sectional view of the torque limiter along lines 13B-13B as depicted in FIG. 13A.

FIG. 14 is a perspective view of a torsion spring.

FIG. 15 is a perspective view of a first rotational body of a rotational assembly of the torque limiter.

FIG. 16 is a perspective view of a second rotational body of the rotational assembly of the torque limiter.

FIG. 17A is a cross-sectional view of the torque limiter generally along the line 17A-17A as depicted in FIG. 13B.

FIG. 17B is a cross-sectional view of the torque limiter generally along the line 17B-17B as depicted in FIG. 13B.

FIG. 18 is a cross-sectional view of the torque limiter disposed within a housing.

FIG. 19 is a cross-sectional view of the torque limiter within a housing taken along the line 19-19 as depicted in FIG. 18.

FIGS. 20A and 20B illustrate approximation of the arms of the torsion spring during twisting of the torsion spring.

FIG. 21A is a cross-sectional view of a proximal end portion of the handle depicting a load cell mounted to the body of the handle.

FIG. 21B is a portion of the handle depicting a plate extension on the gearbox in contact with the load cell.

FIG. 22A is a top view of the gearbox housing.

FIG. 22B is a side view of the gearbox housing.

FIG. 22C is a proximal end view of the gearbox housing.

FIG. 22D is a cross-sectional view of the gearbox housing along line 22D-22D as depicted in FIG. 22B.

FIG. 22E is a cross-sectional view of the gearbox housing along line 22E-22E as depicted in FIG. 22B.

FIG. 22F is a cross-sectional view of the gearbox housing along line 22F-22F as depicted in FIG. 22B.

FIG. 22G is a cross-sectional view of the gearbox housing along line 22G-22G as depicted in FIG. 22B.

FIG. 22H is a cross-sectional view of the gearbox housing along line 22H-22H as depicted in FIG. 22B.

FIG. 22I is a perspective view of a distal end portion of the gearbox housing.

FIG. 22J is a distal end view of the gearbox housing.

FIG. 22K is a perspective view of the gearbox illustrating an encoder mounted on an output shaft.

FIG. 23 is a perspective view of a portion of the handle depicting a pull body coupled to a knob and the gearbox.

FIG. 24A is a perspective view of a portion of the handle depicting a pull body coupled to the gearbox.

FIG. 24B is a perspective view of the pull body viewed from an end of the pull body.

FIG. 24C is a perspective view of the pull body.

FIG. 24D is a cross-sectional view of the pull body along a plane extending along line 24D-24D as depicted in FIG. 24C.

FIG. 24E is a cross-sectional view of a portion of the handle along a plane extending along line 24E-24E as depicted in FIG. 24A with slide arms of the pull body abutting first stop surfaces on the gearbox housing.

FIG. 24F is a cross-sectional view as depicted in FIG. 24E with slider arms of the pull body abutting second stop surfaces on the gearbox housing.

FIG. 24G is a cross-sectional view of a portion of the handle along a plane extending along line 24G-24G as depicted in FIG. 24A.

FIG. 25A is a cross-sectional of a knob having inner channels.

FIG. 25B is a cross-sectional view of the knob along a plane extending along line 25B-25B as depicted in FIG. 25A.

FIG. 26 is a cross-sectional view of the handle along line 26-26 as depicted in FIG. 4A.

### DETAILED DESCRIPTION

### General Considerations

The subject matter is described with implementations and examples. In some cases, as will be recognized by one skilled in the art, the disclosed implementations and examples may be practiced without one or more of the disclosed specific details, or may be practiced with other methods, structures, and materials not specifically disclosed herein. All the implementations and examples described herein and shown in the drawings may be combined without any restrictions to form any number of combinations, unless the context clearly dictates otherwise, such as if the proposed combination involves elements that are incompatible or mutually exclusive. The sequential order of the acts in any process described herein may be rearranged, unless the context clearly dictates otherwise, such as if one act requires the result of another act as input.

In the interest of conciseness, and for the sake of continuity in the description, same or similar reference characters may be used for same or similar elements in different figures, and description of an element in one figure will be deemed to carry over when the element appears in other figures with the same or similar reference character. In some cases, the term “corresponding to” may be used to describe correspondence between elements of different figures. In an example usage, when an element in a first figure is described as corresponding to another element in a second figure, the element in the first figure is deemed to have the characteristics of the other element in the second figure, and vice versa, unless stated otherwise.

The word “comprise” and derivatives thereof, such as “comprises” and “comprising”, are to be construed in an open, inclusive sense, that is, as “including, but not limited to”. The singular forms “a”, “an”, “at least one”, and “the” include plural referents, unless the context dictates otherwise. The term “and/or”, when used between the last two elements of a list of elements, means any one or more of the listed elements. The term “or” is generally employed in its broadest sense, that is, as meaning “and/or”, unless the context clearly dictates otherwise.

The term “coupled” without a qualifier generally means physically coupled or linked and does not exclude the presence of intermediate elements between the coupled elements absent specific contrary language. The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus.

### Intro to the Disclosed Technology

Described herein are prosthetic heart valves, delivery apparatus, and methods for implanting prosthetic heart valves. The prosthetic heart valves can include two or more actuators that can be operated to radially expand or radially compress the prosthetic heart valve. The delivery apparatus can include actuator drivers to releasably engage and operate the actuators.

In some examples, the delivery apparatus can include a counter-rotation mechanism operatively coupled to the actuators such that a net moment force on the prosthetic heart valve while operating the actuators is substantially zero. During expansion of the prosthetic heart valve using the actuators, the counter-rotation movement of the actuators can help maintain the prosthetic heart valve at a rotationally fixed position relative to the native anatomy.

In some examples, the counter-rotation mechanism can include a gearbox pivotably mounted within a handle of the delivery apparatus and coupled to the actuator drivers. In some examples, a stop member can be arranged within the handle to engage and limit pivoting of the gearbox during expansion of the prosthetic heart valve. In some examples, the stop member can include a sensor to measure load on the gearbox while the gearbox is engaged with the stop member.

In some examples, the delivery apparatus can include a mechanism that limits the torque applied to an actuator driver during expansion of the prosthetic heart valve. The torque limiter can be configured to halt a gear train of the gearbox once the torque applied to the actuator driver is within a tolerance of a predetermined maximum torque.

### Examples of the Disclosed Technology

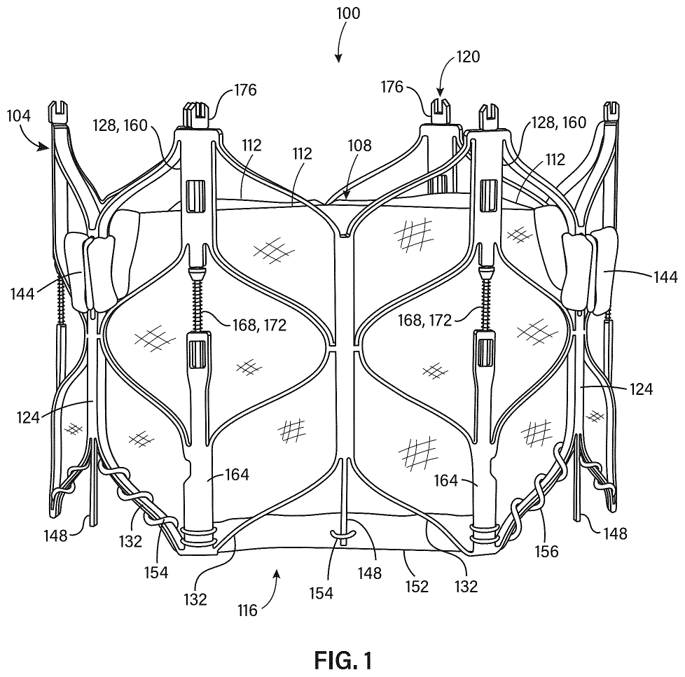

FIG. 1 illustrates a prosthetic heart valve 100, according to one example. The prosthetic heart valve 100 can be configured to replace a native heart valve (e.g., aortic, mitral, pulmonary, and/or tricuspid valves). The prosthetic heart valve 100 is illustrated as a mechanically expandable prosthetic heart valve that can be radially compressed for delivery to an implantation location within a patient's body and then radially expanded to a working diameter at the implantation location. The prosthetic heart valve 100 can include a frame 104 having an annular shape. The prosthetic heart valve 100 can further include a valvular structure 108 supported within and coupled to the frame 104.

In the example, the valvular structure 108 includes one or more leaflets 112 made of flexible material and configured to open and close to regulate blood flow. In one example, the valvular structure 108 can have three leaflets 112, which can be arranged to collapse in a tricuspid arrangement. The leaflets 112 can be made in whole or in part from pericardial tissue (e.g., bovine pericardial tissue), biocompatible synthetic materials, or various other suitable natural or synthetic materials.

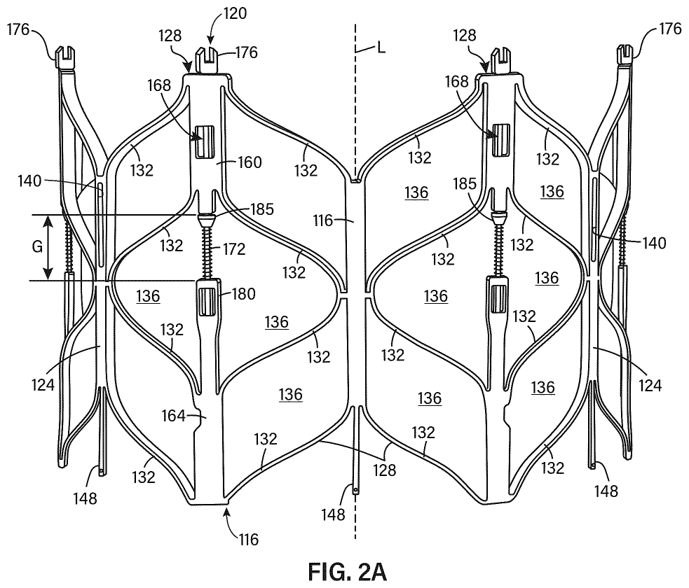

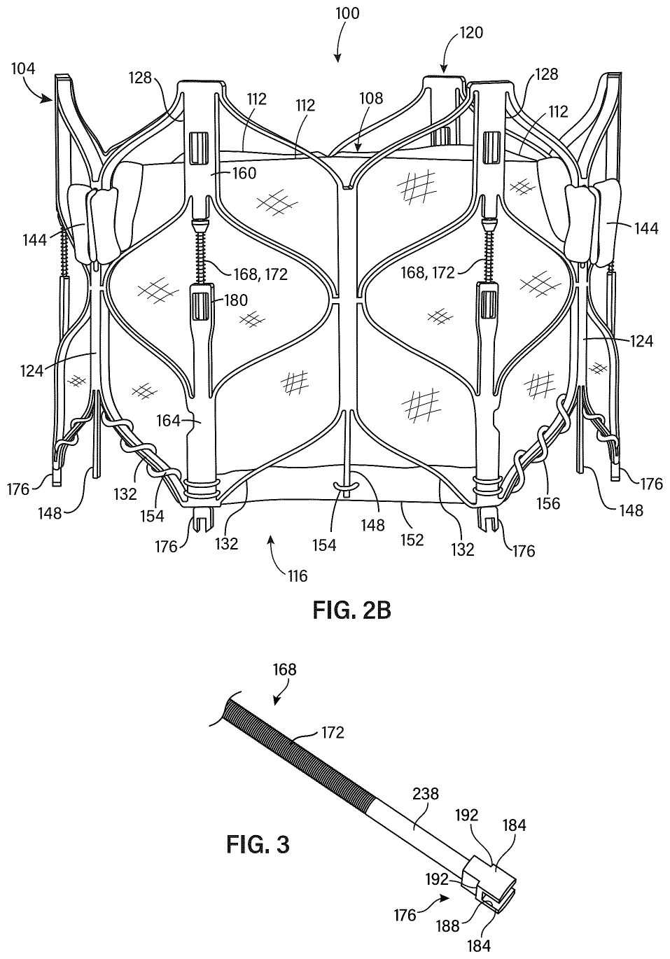

As illustrated more clearly in FIG. 2A, the frame 104 has an inflow end 116, an outflow end 120, and a longitudinal axis L extending in a direction from the inflow end 116 to the outflow end 120. The frame 104 can include a plurality of support posts 124, 128 aligned with the longitudinal axis L and spaced along a circumference of the frame 104. In one example, the support posts 124, 128 can be arranged in an alternating manner along the circumference of the frame 104. The frame 104 can further include a plurality of struts 132 extending circumferentially between adjacent support posts 124, 128 and interconnecting the support posts 124, 128. The struts 132 and support posts 124, 128 define cells 136 of the frame 104. As illustrated, the struts 132 can have a curved shape.

As illustrated in FIGS. 1 and 2A, one or more commissure windows 140 can be formed in one or more of the support posts 124. Commissures 144 can be formed at the commissure windows 140 to couple the leaflets 112 to the frame 104. One or more of the support posts 124 can further include cantilevered struts 148 extending to the inflow end 116 of the frame 104. In some cases, inflow edge portions 152 of the leaflets 112 can be attached to the cantilevered struts 148 (e.g., by sutures 154) and/or to selected struts 132 of the frame 104 (e.g., using sutures 156).

In one example, the frame 104 can be adjusted between a radially expanded configuration and a radially compressed configuration by deflecting the struts 132. In one example, the frame 104 (e.g., the posts and struts) can be made of biocompatible plastically-expandable materials that will allow the frame 104 to be adjusted between the radially expanded configuration and radially compressed configuration. Suitable examples of plastically-expandable materials that can be used in forming the frame 104 include, but are not limited to, stainless steel, cobalt chromium alloy, and/or nickel titanium alloy (which can also be referred to as “NiTi” or “nitinol”).

Referring to FIG. 2A, in one example, one or more actuators 168 can be coupled to the support posts 128 and used to adjust the frame 104 between the radially expanded configuration and the radially compressed configuration. In one example, each support post 128 can include an upper post member 160 and a lower post member 164 (the terms “upper” and “lower” are relative to the orientation of the frame 104 in FIG. 1) aligned with the longitudinal axis L of the frame 104 and having opposing ends separated by a gap G. The respective actuator 168 can be coupled to the post members 160, 164 and operable to increase or decrease the gap G in order to radially compress or expand the frame 104.

In one example, the actuator 168 can include an actuator rod 172 with an attached actuator head 176. In the examples illustrated in FIGS. 2A and 2B, the actuator rod 172 extends through or into the post members 160, 164 and across the gap G. In the example illustrated in FIG. 2A, the actuator rod 172 is inserted into the upper post member 160 from the outflow end 120, and the actuator head 176 is disposed or retained at the outflow apex of the upper post member 160. In the example illustrated in FIG. 2B, the actuator rod 172 is inserted into the lower post member 164 from the inflow end 116, and the actuator head 176 is disposed or retained at the inflow apex of the lower post member 164.

In some examples, the actuator rod 172 is externally threaded. As illustrated in FIGS. 2A and 2B, the lower post member 164 can include a nut 180 with an internal thread to threadedly engage the actuator rod 172. In this case, the actuator rod 172 can be translated in a longitudinal direction by rotating the actuator rod 172 relative to the nut 180. In some examples, the actuator rod 172 can be freely slidable relative to the upper post member 160. In other examples, the actuator rod 172 can threadedly engage the upper post member 160.

As illustrated in FIG. 3, the actuator head 176 can include a pair of protrusions 184 forming a slot 188. The actuator head 176 can further include one or more shoulders 192. As will be further described, an actuation assembly of the delivery apparatus can releasably engage the actuator head 176 via the slot 188 and shoulders 192.

Referring to FIGS. 2A and 2B, in one scenario, the actuator rod 172 can be rotated in a first direction to move the upper post member 160 towards the lower post member 164 and thereby decrease the size of gap G, which can have the effect of radially expanding the frame 104. In another scenario, the lower post member 164 may be held steady while the actuator rod 172 is rotated in a second direction to move the upper post member 160 away from the lower post member 164 and thereby increase the size of gap G, which can have the effect of radially compressing the frame 104. To avoid over-crimping the prosthetic heart valve, a stopper 185 (e.g., a nut) may be installed on the actuator rod 172 to limit the travel of the actuator rod 172 while rotating the actuator rod 172 to radially compress the frame 104.

In an alternative implementation, as will be further described, some of the actuator rods 172 can be rotated in one direction while the other actuator rods 172 are rotated in an opposite direction simultaneously to either radially expand the frame or radially compress the frame. This counter-rotation of the actuator rods can be used to help reduce the likelihood of the entire frame 104 rotating about the longitudinal axis L during rotation of the actuator rods 172 about their respective axes (e.g., when radially expanding the frame 104).

Additional examples of mechanically expandable valves can be found in International Application No. PCT/US2021/052745 and U.S. Provisional Application No. 63/209,904, which are incorporated by reference herein.

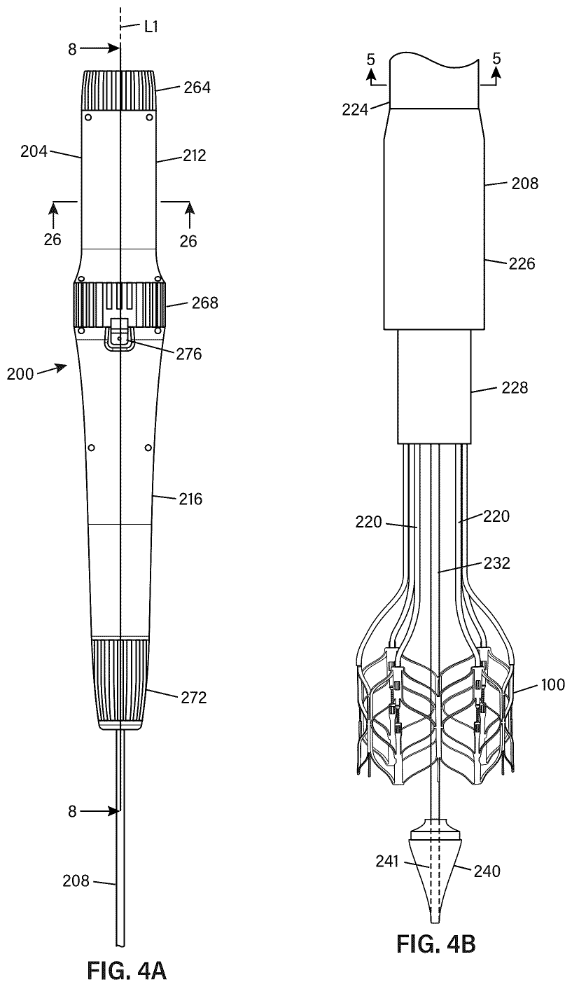

FIGS. 4A and 4B illustrate a delivery apparatus 200, according to one example. The delivery apparatus 200 can be used to deliver the prosthetic heart valve 100 to an implantation location within a patient's body. The delivery apparatus 200 includes a handle 204 and a shaft assembly 208 coupled to the handle 204. The delivery apparatus 200 can further include one or more actuation assemblies 220 that can be used to releasably couple the prosthetic heart valve 100 to a distal end portion of the shaft assembly 208 and to radially expand and/or compress the prosthetic heart valve 100.

The prosthetic heart valve 100 is shown in an expanded configuration in FIG. 4B. To facilitate delivery of the prosthetic heart valve 100 to an implantation location, the delivery apparatus 200 (and/or other crimping devices) can be used to move the prosthetic heart valve 100 from a radially expanded, functional configuration to a radially compressed, delivery configuration. Once at the implantation location, actuation drivers of the actuation assemblies 220 can operate the actuators 168 of the prosthetic heart valve 100 to radially expand the prosthetic heart valve 100 to a working diameter.

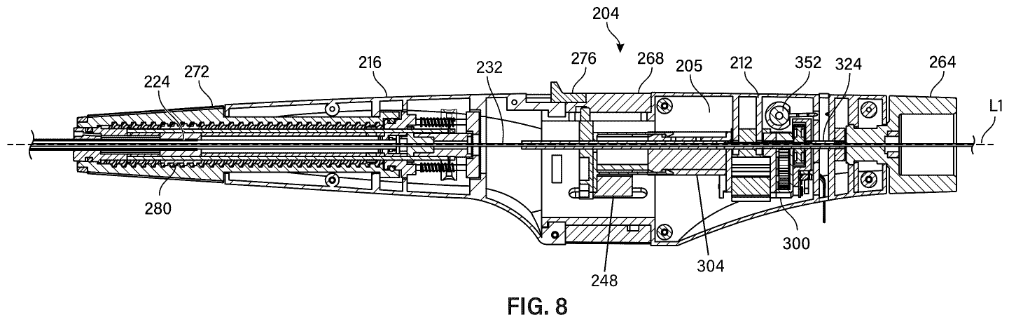

In one example, the handle 204 includes a proximal body portion 212 and a distal body portion 216 coupled together. The body portions 212, 216 define a cavity (depicted as 205 in FIG. 8) extending along a longitudinal axis L1 of the handle 204. Various mechanisms of the delivery apparatus 200 are disposed within the cavity 205.

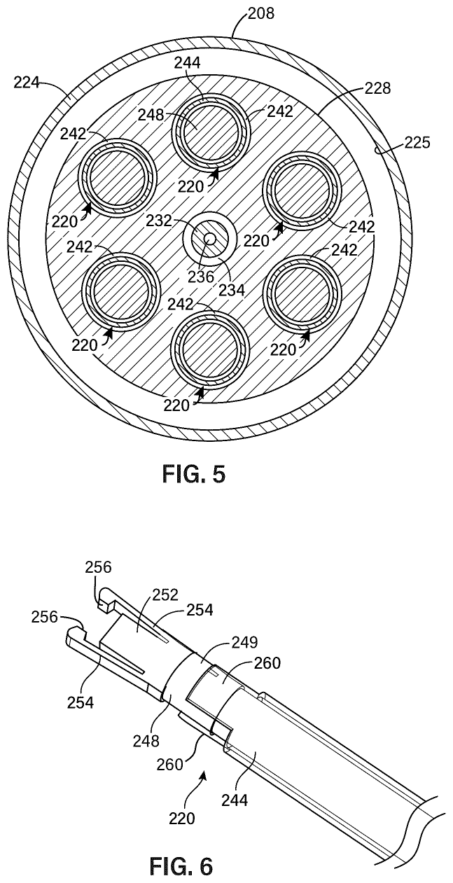

As illustrated in FIGS. 4B and 5, the shaft assembly 208 can include an outer delivery shaft 224 having a lumen 225 extending along the entire length of the shaft. The shaft assembly 208 can include a multi-lumen delivery shaft 228 extending through the lumen 225 and having lumens 234, 242. The shaft assembly 208 can include a nosecone shaft 232 extending through the lumen 234. The actuation assemblies 220 can extend through the lumens 242. The lumen 234 can be centrally disposed within the multi-lumen delivery shaft 228, and the lumens 242 can be angularly spaced apart (uniformly or non-uniformly) about a central axis of the multi-lumen delivery shaft 228 and disposed around the lumen 234.

In one example, the proximal end portion of the nosecone shaft 232 extends into the portion of the cavity of the handle 204 defined in the proximal body portion 212 (indicated in FIG. 4A), and the distal end portion of the nosecone shaft 232 extends distally from the distal end of the multi-lumen delivery shaft 228 (as shown in FIG. 4B). The prosthetic heart valve 100 can be disposed around the distal end portion of the nosecone shaft 232 when releasably coupled to the actuation assemblies 220.

The nosecone shaft 232 can define a guidewire lumen 236 for receiving a guidewire. As shown in FIG. 4B, a nosecone 240 can be attached to a distal end of the nosecone shaft 232. The nosecone 240 can have a central opening 241 that is aligned and connected to the guidewire lumen 236. During an implantation procedure, a guidewire can be initially inserted into a patient's vasculature. The proximal end of the guidewire can be inserted into the central opening 241 of the nosecone 240 to allow the delivery apparatus 200 to be advanced through the patient's vasculature to an implantation location over the guidewire.

FIG. 6 illustrates a distal end portion of the actuation assembly 220. Each actuation assembly 220 can include an outer sleeve 244 and an actuator driver 248 extending through the outer sleeve 244. In the example, the actuator driver 248 includes a distal head having a central protrusion 252 and one or more flexible elongated elements 254. The central protrusion 252 can be configured to extend into the slot 188 (shown in FIG. 3) of the actuator head 176 of an actuator 168 of the prosthetic heart valve. The flexible elongated elements 254 can have radial protrusions 256 configured to engage the shoulders 192 (shown in FIG. 3) of the actuator head 176.

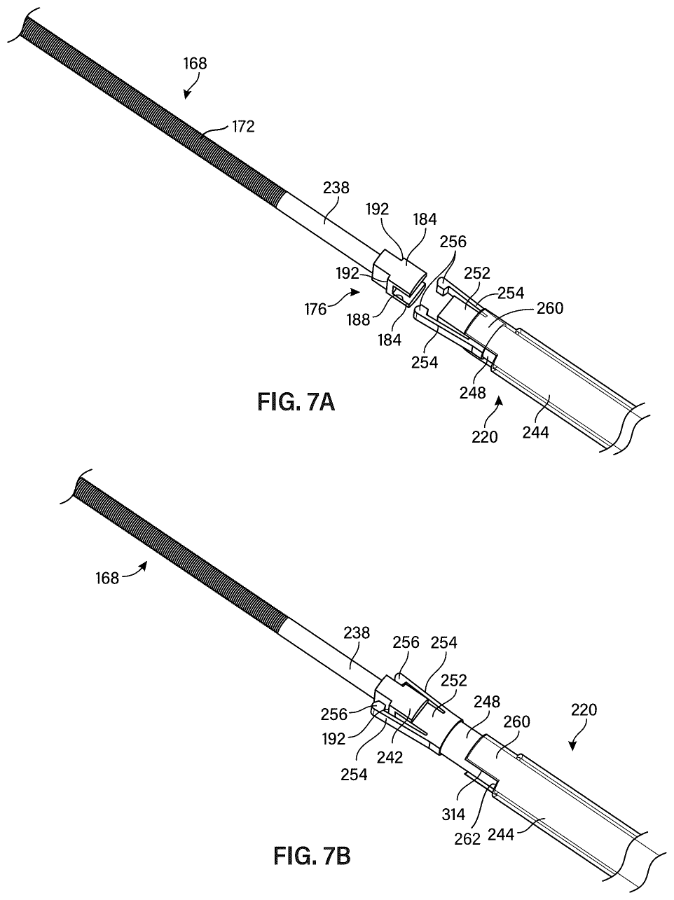

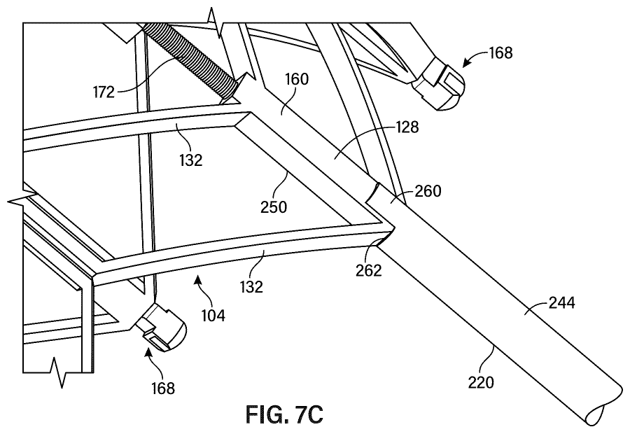

FIGS. 7A-7C illustrate engagement of an actuation assembly 220 with a respective actuator 168. Initially, the distal end portion of the actuation assembly 220 is aligned with the actuator head 176 of the actuator 168, as shown in FIG. 7A. The distal end portion of the actuator driver 248 is then advanced such that the central protrusion 252 of the actuator driver 248 is disposed within the slot 188 of the actuator head 176 of the actuator 168. When the central protrusion 252 is engaged with the slot 188, the flexible elongated elements 254 are disposed at the sides of the actuator head 176, and the radial protrusions 256 of the flexible elongated elements 254 are positioned distally to the shoulders 192 on the actuator head 176, as shown in FIG. 7B.

The outer sleeve 244 can be advanced over the distal end portion of the actuator driver 248 to radially compress the flexible elongated elements 254 against the actuator head 176 until the radial protrusions 256 abut the shoulders 192, thereby coupling the actuator driver 248 to the actuator 168. The outer sleeve 244 can be further advanced until the outer sleeve 244 engages the frame 104, as illustrated in FIG. 7C.

The outer sleeve 244 can have first and second support extensions 260 defining gaps or notches 262 between the extensions 260. As illustrated in FIG. 7C, the support extensions 260 can be oriented such that when the actuation assembly 220 is coupled to a respective actuator 168, the support extensions 260 extend partially over a proximal end portion of the upper post arm 160 of the respective support post 128. The engagement of the support extensions 260 with the frame 104 can counteract rotational forces applied to the frame 104 by the actuator rods 172 during expansion of the frame 104.

Various other coupling mechanisms can be used to releasably couple the prosthetic heart valve to the actuation assembly of the delivery apparatus. For example, additional coupling mechanisms are described in U.S. Application No. 63/194,285 and U.S. patent application having attorney docket no. 12052US01, which are incorporated by reference herein.

As shown in FIG. 4A, the handle 204 can include one or more knobs that can be configured to perform various functions of the delivery apparatus 200 to deliver the prosthetic heart valve 100 to an implantation location within a patient's body. In one example, the handle 204 can include a first knob 264, a second knob 268, and a third knob 272. In one example, the knobs 264, 268, 272 can be knobs that are rotatable about the longitudinal axis L1 of the handle 204 and relative to the body portions 212, 216 of the handle. The handle 204 can include other knobs that can be rotatable or slidable, such as a safety knob 276.

In the example, the first knob 264 is located at a proximal end of the handle 204 and can be used to operate the actuation assemblies 220 of the delivery apparatus 200 and the actuators 168 of the prosthetic heart valve 100. As illustrated in FIG. 8, the first knob 264 can be configured to operate a gearbox 300 disposed within a proximal portion of the cavity 205 of the handle 204. The actuator drivers 248 of the actuation assemblies 220 can be coupled to the gearbox 300 in order to be rotated by the gearbox 300. The rotation of the actuator drivers 248 can be translated to rotational motion of the actuators 168 of the prosthetic heart valve 100.

In the example, the second knob 268 is located where the proximal and distal body portions 212, 216 of the handle 204 are coupled together. The second knob 268 can be configured to release the actuation assemblies 220 from the prosthetic heart valve 100 (e.g., after positioning the prosthetic heart valve 100 at the desired implantation location and expanding the prosthetic heart valve 100 to the working diameter). In one example, the safety knob 276 can be configured to prevent unintentional release of the actuation assemblies 220 from the prosthetic heart valve. For example, the safety knob 276 can slide into a recess in the second knob 268 to prevent rotation of the second knob 268. Retraction of the safety knob 276 from the recess can allow the second knob 268 to be rotated.

In the example, the third knob 272 is located at a distal end of the handle 204. The third knob 272 can be configured such that rotation of the knob relative to the handle body results in the outer delivery shaft 224 moving axially relative to the actuation assemblies 220, the prosthetic heart valve 100, and the nosecone shaft 232.

In one example, a delivery capsule 226 (shown in FIG. 4B) can be attached to a distal end of the outer delivery shaft 224. Axial movement of the outer delivery shaft 224 in a distal direction relative to the other shafts and prosthetic valve can move the delivery capsule 226 over the distal end portions of the actuation assemblies 220 and the prosthetic heart valve 100 (i.e., when the prosthetic heart valve 100 is in the radially compressed configuration) such that the prosthetic heart valve 100 is enclosed within the delivery capsule. Axial movement of the outer delivery shaft 224 in a proximal direction relative to the other shafts and the prosthetic valve can retract the delivery capsule 226 from the prosthetic heart valve 100, exposing the prosthetic heart valve, for example, for deployment at an implantation location. In some examples, the third knob 272 can be operatively coupled to a carriage 280 within the distal portion of the cavity 205 of the handle 204. The outer delivery shaft 224 can be coupled to the carriage 280 such that movement of the carriage 280 due to rotation of the third knob 272 results in axial displacement of the outer delivery shaft 224.

During expansion of the prosthetic heart valve 100, rotation of the actuators 168 can apply moment forces to the frame 104, i.e., due to the frictional forces acting between the frame 104 and the actuator rods 172 of the actuators 168. These moment forces can, in some instances, result in the frame 104 rotating or pivoting about the longitudinal axial L of the frame during the expansion/contraction procedure. To help reduce such rotation of the entire frame, the actuators 168 can be divided into two sets, and the two sets can be rotated in opposite directions such that the moment forces due to one set of actuators is counterbalanced by the moment forces due to the other set of actuators. This can, for example, help the frame 104 to remain rotationally fixed or at least substantially rotationally fixed during expansion of the prosthetic heart valve. Thus, this configuration can, for example, make positioning and/or deploying a prosthetic heart valve relatively easier and/or predictable.

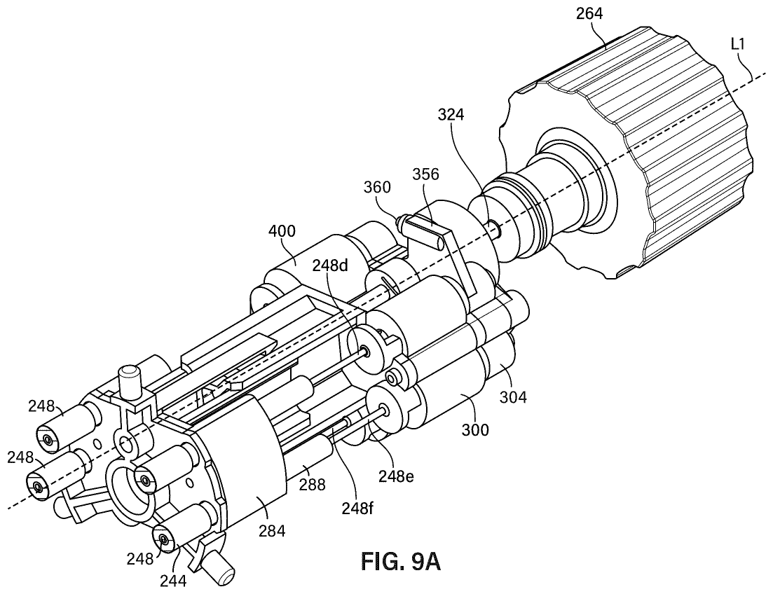

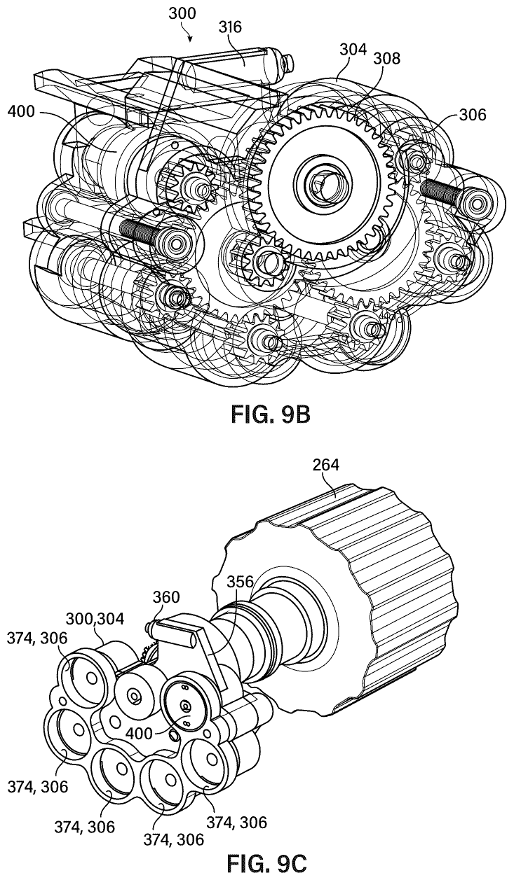

As illustrated in FIGS. 9A-9C, the gearbox 300 of the handle 204 can include a gearbox housing 304 with various compartments 306 to hold the components of a gear train 308. The output shafts of the gear train 308 can be coupled to the actuator drivers 248 such that operation of the gear train 308 results in rotation of the actuator drivers 248 and consequently rotation of the actuators 168 of the prosthetic heart valve 100. In one example, the gearbox 300 can be a counter-rotation gearbox where the gear train 308 is configured to rotate two sets of actuator drivers in opposite directions.

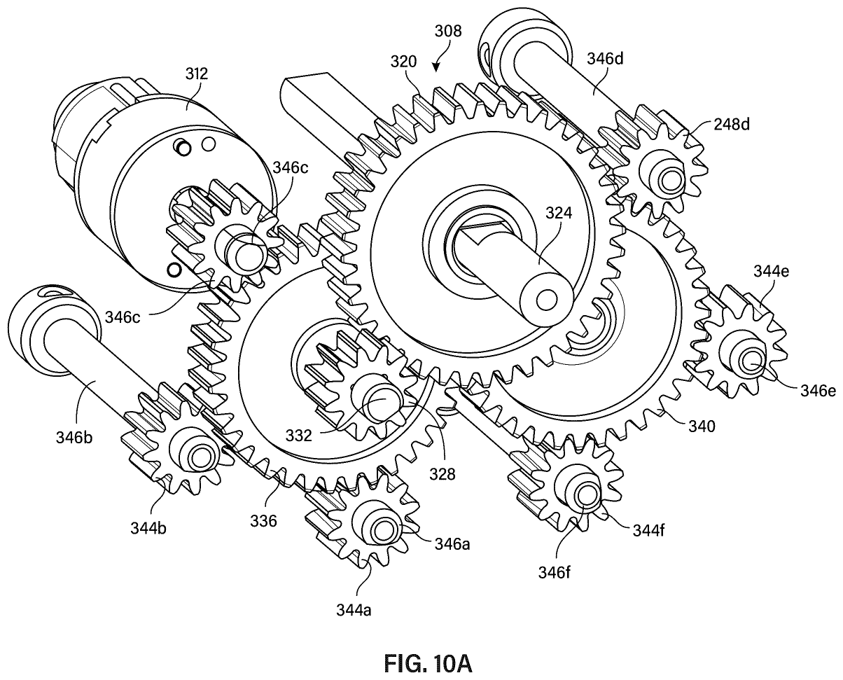

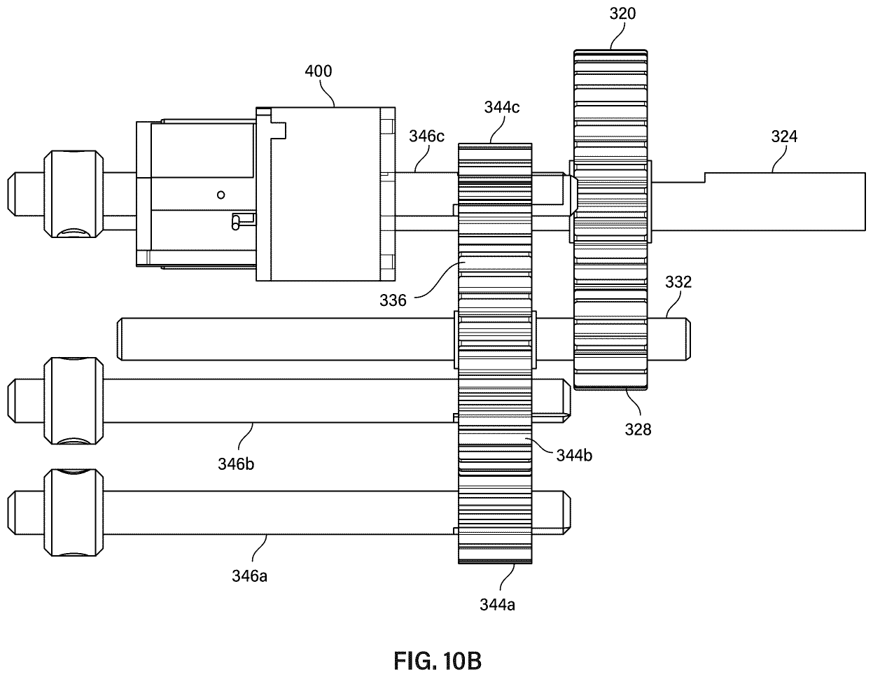

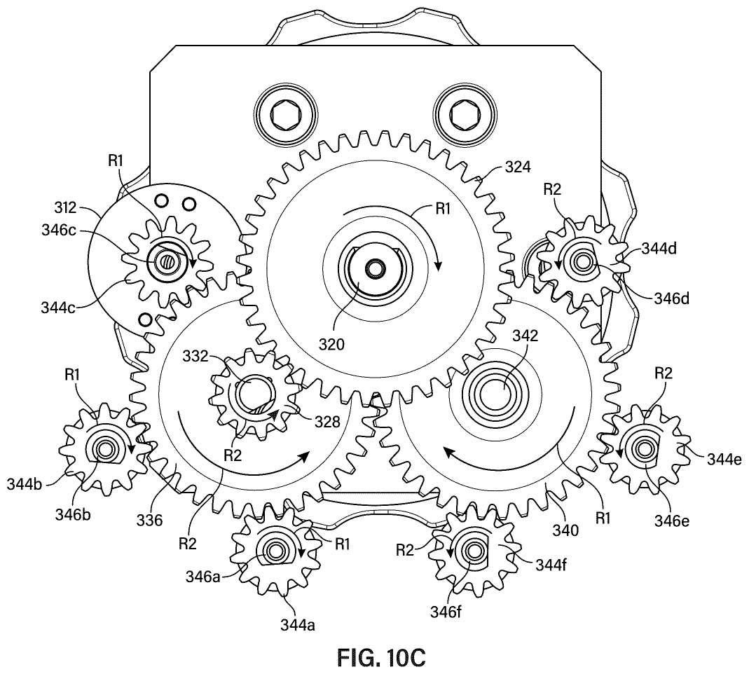

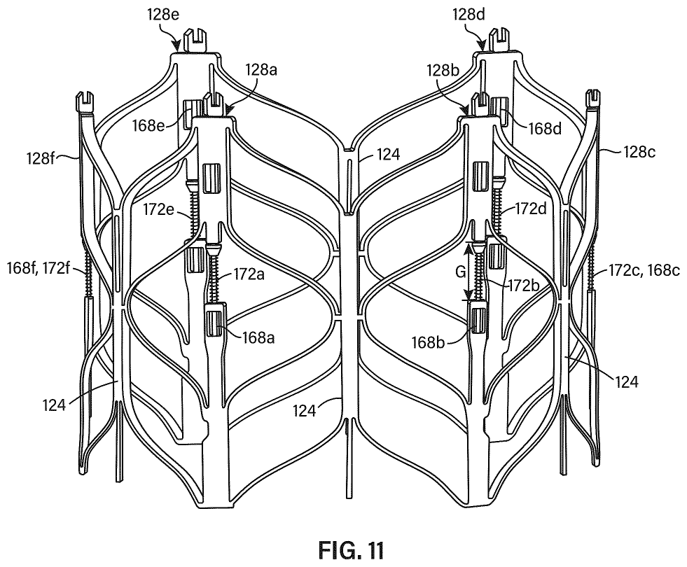

FIGS. 10A-10C illustrate one implementation of the gear train 308. In the example, the gear train 308 includes an input shaft 324 and an input gear 320 coupled to the input shaft 324. In one example, the input shaft 324 is aligned with the longitudinal axis L1 of the handle 204 (as depicted in FIG. 8). The input shaft 324 can be coupled to the first knob 264 of the handle 204 (as depicted in FIG. 8) such that rotation of the first knob 264 results in rotation of the input shaft 324. The input gear 320 rotates with the input shaft 324. The rotational direction R1 of the input gear 320 can be clockwise or counterclockwise, depending on the direction in which the first knob 264 is rotated.

The gear train 308 can include a transmission gear 328 coupled to a transmission shaft 332, which can be arranged in parallel to the input shaft 324. The teeth of the input gear 320 are meshed with the teeth of the transmission gear 328 such that rotation of the input gear 320 drives the transmission gear 328. The transmission shaft 332 rotates with the transmission gear 328. In one example, rotation of the input gear 320 in a first direction R1 drives the transmission gear 328 in a second direction R2 that is opposite to the first direction (whether R2 is clockwise or counterclockwise will depend on the rotational direction R1 as determined by the rotation of the first knob 264).

The gear train 308 can include a first driving gear 336 coupled to the transmission shaft 332 and disposed distally to the transmission gear 328. In this case, rotation of the transmission shaft 332 in response to driving the transmission gear 328 by the input gear 320 is translated to rotation of the first driving gear 336. The first driving gear 336 rotates in the same direction R2 as the transmission gear 328.

The gear train 308 can include a second driving gear 340 supported on a driving shaft 342 that is arranged in parallel to the transmission shaft 332. The teeth of the second driving gear 340 are meshed with the teeth of the first driving gear 336 such that rotation of the first driving gear 336 drives the second driving gear 340. The driving shaft 342 rotates with the second driving gear 340. The second driving gear 340 rotates in a direction R1 that is opposite to the direction R2 in which the first driving gear 336 rotates.

The gear train 308 can include a set of first output gears (which can also be referred to as “pinion gears”) angularly spaced apart about a central axis of the first driving gear 336 and having teeth meshed with the teeth of the first driving gear 336. In the example, the set of first output gears includes output gears 344a, 344b, 344c. The output gears 344a, 344b, 344c rotate in a direction R1 that is opposite to the direction R2 in which the first driving gear 336 is rotating. In one example, the output gears 344a, 344b, 334c are coupled to output shafts 346a, 346b, 346c, respectively. The output shafts 346a, 346b, 346c can be coupled to a first set of actuator drivers.

The gear train 308 can include a second set of output gears (which can also be referred to as “pinion gears”) angularly spaced apart about a central axis of the second driving gear 340 and having teeth meshed with the teeth of the second driving gear 340. In the example, the second set of output gears includes output gears 344d, 344e, 344f. The output gears 344d, 344e, 344f rotate in a direction R2 that is opposite to the direction R1 in which the second driving gear 340 is rotating. As such, the output gears 344d, 344e, 344f of the second set of output gears rotate in a direction that is opposite to the direction in which the output gears 344a, 344b, 344c of the first set of output gears rotate. In one example, the output gears 344d, 344e, 344f are coupled to output shafts 346d, 346e, 346f, respectively. The output shafts 346d, 346e, 346f can be coupled to a second set of actuator drivers.

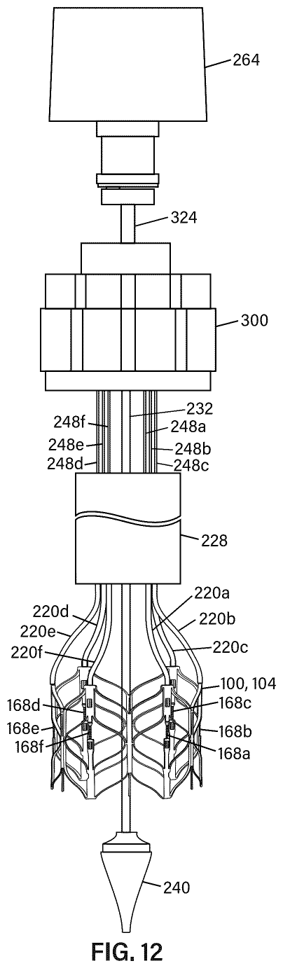

For illustrative purposes, FIG. 11 shows the frame 104 with actuators 168a, 168b, 168c, 168d, 168e, 168f coupled to support posts 128a, 128b, 128c, 128d, 128e, 128f, respectively. In one example, a first set of actuators can include actuators 168a, 168b, 168c, and a second set of actuators can include actuators 168d, 168e, 168f. The first set of actuators 168a, 168b, 168c can be coupled to the first set of actuator drivers 248a, 248b, 248c, and the second set of actuators 168d, 168e, 168f can be coupled to the second set of actuator drivers 248d, 248e, 248f, as illustrated in FIG. 12 (several details of the delivery apparatus are not shown in FIG. 12 for simplicity; for example, the body of the handle 204 and the outer delivery shaft 224 through which the multi-lumen delivery shaft 228 extends are not shown).

Returning to FIG. 11, the actuator rods 172a, 172b, 172c of the actuators 168a, 168b, 168c in the first set of actuators can have threads with a first configuration (e.g., right-hand threads). The actuator rods 172d, 172e, 172f of the actuators 168d, 168e, 168f in the second set of actuators can have threads with a second configuration (e.g., left-hand threads) that are opposite to the first configuration. For example, if the actuator rods 172a, 172b, 172c have right-hand threads, the actuator rods 172d, 172e, 172f can have left-hand threads (or vice versa). Thus, when the actuators 168a, 168b, 168c in the first set and the actuators 168d, 168e, 168f in the second set are simultaneously rotated in opposite directions, all the actuators will act in concert to either increase the respective gap G or decrease the gap G.

Other examples of dividing the actuators into two sets are possible. For example, a first set of actuators could include actuators 168a, 168c, 168e, and a second set of actuators could include actuators 168b, 168d, 168f (i.e., alternating actuators around the circumference of the frame could be included in a set). In this case, the actuator rods 172a, 172c, 172e of the first set of actuators can have threads with a first configuration (e.g., right-hand threads), and the actuator rods 172b, 172d, 172f of the second set of actuators can have threads with a second configuration that is opposite to the first configuration (e.g., left-hand threads).

Examples have been given with the prosthetic heart valve 100 having six actuators divided into two sets. In other examples, the prosthetic heart valve could have greater than six (e.g., 7-15) or fewer than six (e.g., 1-5) actuators. In other cases, the prosthetic heart valve could have an odd number of actuators, in which case one set of actuators could have a greater number of actuators compared to the other set of actuators. The number of actuation assemblies/actuator drivers of the delivery apparatus can generally match the number of actuators of the prosthetic heart valve.

In the simplified illustration of FIG. 12, each of the actuator drivers 248a, 248b, 248c in the first set of actuator drivers extends through the multi-lumen delivery shaft 228 and is connected to a respective actuator 168a, 168b, 168c of the prosthetic heart valve 100. Similarly, each of the actuator drivers 248d, 248e, 248f in the second set of actuator drivers extends through the multi-lumen delivery shaft 228 and is connected to a respective actuator 168d, 168e, 168f of the prosthetic heart valve 100. The actuator drivers 248a, 248b, 248c are coupled to the first set of output shafts of the gearbox 300 (346a, 346b, 346c in FIGS. 10A-0C), and the actuator drivers 248d, 248e, 248f are coupled to the second set of output shafts of the gearbox 300 (346d, 346e, 346f in FIGS. 10A-10C). The input shaft 324 of the gearbox 300 is coupled to the first knob 264.

To radially expand the prosthetic heart valve 100, for example, at an implantation location, the first knob 264 can be used to rotate the first set of actuator drivers 248a, 248b, 248c and the second set of actuator drivers 248d, 248e, 248f in opposite directions. The counter-rotation of the two sets of actuator drivers results in counter-rotation of the first set of actuators 168a, 168b, 168c and the second set of actuators 168d, 168e, 168f. This counter-rotation of the two sets of actuators can advantageously help reduce the likelihood of the prosthetic heart valve rotating relative to the native anatomy during expansion of the prosthetic heart valve.

In some implementations, a torque limit can be defined for each actuator driver 248, and one or more torque limiters can be provided (e.g., one for each actuator driver 248) to prevent torque on the actuator driver 248 from exceeding the predefined limit. The torque limiter can, for example, prevent overloading of the actuator driver 248 during expansion of the prosthetic heart valve 100. In one example, the torque limiter restricts rotation of the corresponding actuator driver 248 when the torque on the actuator driver 248 has reached a predefined limit. Since all the actuator drivers 248 are coupled to the gear train 308, the gear train 308 effectively halts when any of the actuator drivers 248 is stopped by the torque limiter.

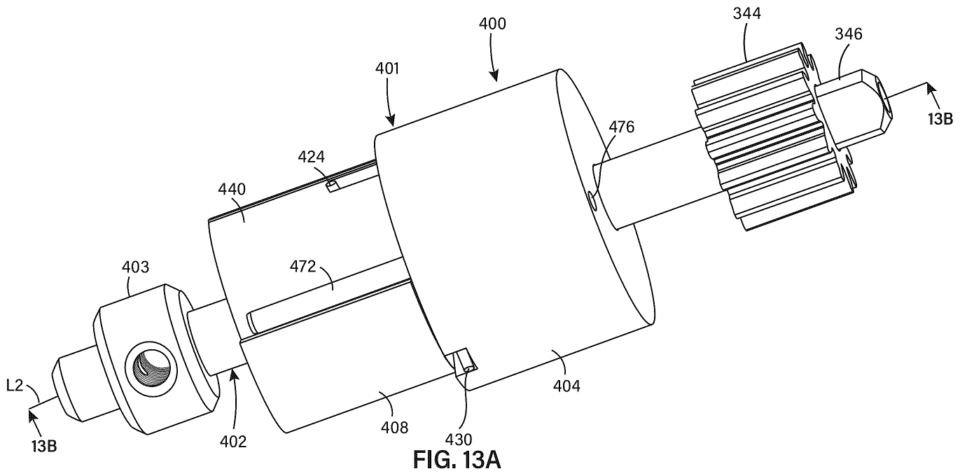

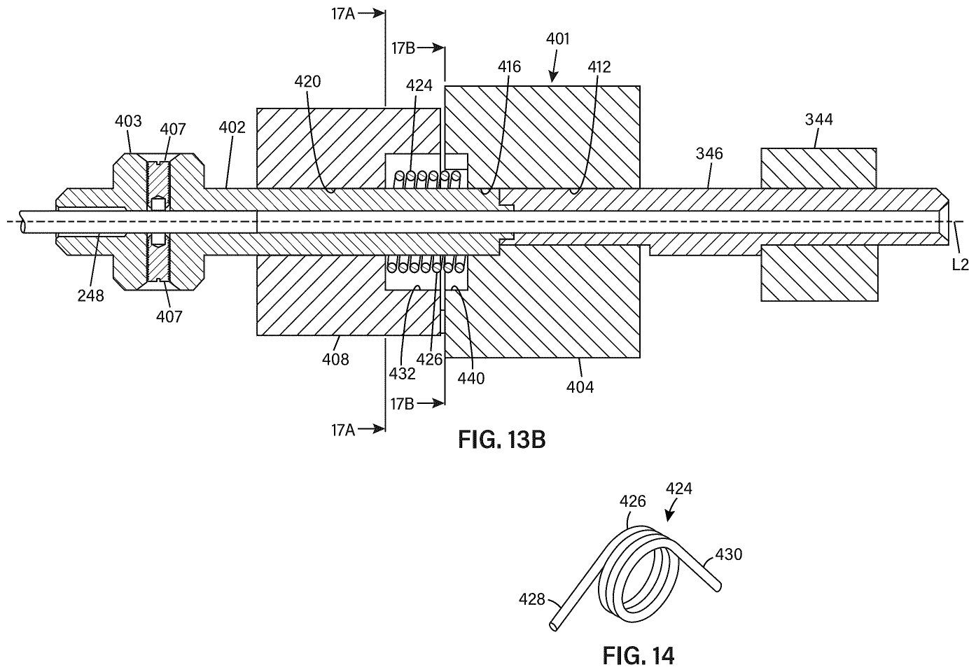

FIGS. 13A and 13B illustrate a torque limiter 400, according to one example. The torque limiter 400 can couple an actuator driver 248 to an output shaft 346 of the gear train 308 and can operate to prevent rotation of the actuator driver 248 when a torque on the actuator driver 248 is within a predetermined torque limit range. The upper limit of the predefined torque limit range can be a maximum torque on the actuator driver 248, and the lower limit of the predetermined torque limit range can be a torque that is within a tolerance of the maximum torque (e.g., within 15% of the maximum torque). In one example, the maximum torque on the actuator driver 248 can be 50 N-mm. The torque limiter 400 can be housed within a compartment of the gearbox housing 304. For illustrative purposes, FIG. 9B shows the torque limiter 400 within one of the compartments 306 of the gearbox housing 304. Although only one torque limiter 400 is depicted in FIGS. 9B and 9C, in some examples, the handle 204 for the delivery apparatus 200 can comprise a plurality (e.g., 2-15) of torque limiters 400. For example, each actuation driver 248 of the delivery apparatus could have a respective torque limiter 400.

Returning to FIGS. 13A-13B, the torque limiter 400 has a longitudinal axis L2. The torque limiter 400 includes a rotatable assembly 401 aligned with and rotatable about the longitudinal axis L2. The rotatable assembly 401 couples a connector shaft 402 to one of the output shafts 346 of the gearbox 300. An output gear 344 is coupled to the output shaft 346, as previously described. One of the actuator drivers 248 can be coupled to the connector shaft 402 at a coupling section 403 of the connector shaft 402 (e.g., using one or more set screws 407). In one mode, the rotatable assembly 401 allows the connector shaft 402 to rotate with the output shaft 346. In another mode, the rotatably assembly 401 prevents rotation of both the connector shaft 402 and the output shaft 346.

The rotatable assembly 401 includes a first rotatable body 404 and a second rotatable body 408. In the example, the second rotatable body 408 is positioned distally to the first rotatable body 404, and both the first and second rotatable bodies 404, 408 are rotatable about the longitudinal axis L2. The first rotatable body 404 is fixedly coupled to the output shaft 346 such that the first rotatable body 404 and the output shaft 346 can rotate together about the longitudinal axis L2. In the example, the first rotatable body 404 is positioned distally to the output gear 344. The second rotatable body 408 is fixedly coupled to the connector shaft 402 such that the second rotatable body 408 and the connector shaft 402 can rotate together about the longitudinal axis L2.

In one example, the first rotatable body 404 includes a proximal axial bore 412 and a distal axial bore 416. A distal end portion of the output shaft 346 is inserted into the proximal axial bore 412 and engages the proximal axial bore 412 in a manner that allows the first rotatable body 404 to rotate with the output shaft 346. In one example, the proximal axial bore 412 can have a non-circular cross-sectional profile (taken in a plane perpendicular to the longitudinal axis L2) that is adapted to match with a non-circular cross-sectional profile (taken in a plane perpendicular to the longitudinal axis L2) on the input shaft 346 such that rotation of the input shaft 346 results in rotation of the first rotatable body 404. For example, the non-circular cross-sectional profile of the proximal axial bore 412 can be “D shaped” (which can also be referred to as having a “flat”) that can engage a similarly D-shaped (or “flat”) output shaft 346 and allow the first rotatable body 404 to rotate in the same direction as the output shaft 346. Alternatively, the output shaft 346 can be attached to the proximal axial bore 412 (e.g., by other means for fixedly coupling such as welding, gluing, and the like) to allow the first rotatable body 404 to rotate with the output shaft 346.

The second rotatable body 408 can include an axial bore 420 that is aligned with the distal axial bore 416 of the first rotatable body 404. The connector shaft 402 extends through the axial bore 420 of the first rotatable body 404 into the distal axial bore 416 of the first rotatable body 404. The connector shaft 402 can engage the second rotatable body 408 in a manner that allows the connector shaft 402 to rotate with the second rotatable body 408. For example, the axial bore 420 can have a non-circular profile to engage a complementary non-circular profile on the connector shaft member 402. Alternatively, the connector shaft 402 can be attached to the axial bore 420 (e.g., by welding, gluing, and the like) to allow the second rotatable body 408 to be rotatable with the connector shaft 402. In some examples, the distal end of the output shaft 346 and the proximal end of the connector shaft 402 can be axially spaced apart (e.g., separated by a wall or shoulder of the first rotatable body 404).

In other examples, the opposing ends of the connector shaft 402 and output shaft 346 can axially overlap. In such example, the shafts 402, 346 can include one or more features that facilitate alignment of the connector shaft 402 with the output shaft 346 along the longitudinal axis L2 while also allowing relative rotational movement between the connector shaft 402 and the input shaft 346. For example, in some instances, the connector shaft 402 (or at least a portion thereof) can comprise an outer diameter that is smaller than a diameter of an internal bore of the output shaft 346 such that the connector shaft can extend axially into the output shaft 346 (or vice versa).

In any event, the output shaft 346 and the connector shaft 402 are not fixedly coupled together. Thus, in some instances, which are further explained below, the output shaft 346 (and the first rotatable body 404) and the connector shaft 402 (and the second rotatable body 408) can rotate relative to each other.

In the example, the first rotatable body 404 and the second rotatable body 408 are coupled together by a rotational biasing member (e.g., a torsion spring 424). As illustrated in FIG. 14, the torsion spring 424 can be a helical torsion spring including a coil portion 426 terminating at opposite ends in first and second end (or arm) portions 428, 430. The first and second end portions 428, 430 of the torsion spring 424 can extend radially outward beyond the coil portion 426 of the torsion spring 424. The torsion spring 424 can be configured such that the first end portion 428 is rotationally offset from the second end portion 430.

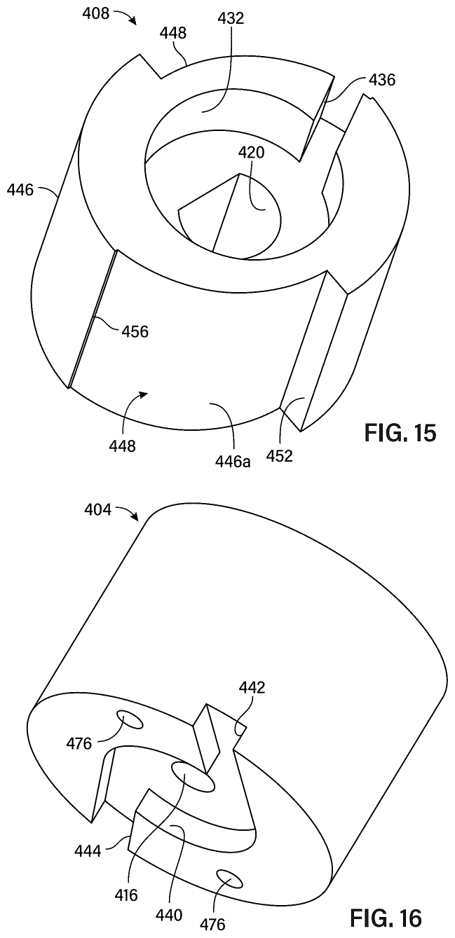

In one example, as illustrated in FIG. 15, the proximal end portion of the second rotatable body 408 can include a recess 432 and a connected lateral slot 436. The recess 432 can be centrally aligned with the longitudinal axis L2 and connected to the axial bore 420. As illustrated in FIG. 16, the distal end portion of the first rotatable body 404 can include a recess 440 and connected lateral slots 442, 444. The recess 440 can be centrally aligned with the longitudinal axis L2 and connected to the distal axial bore 416. The lateral slots 442, 444 are rotationally offset from each other about the longitudinal axis L2. As shown in FIG. 13B, the connector shaft 402 can extend through the recesses 432, 440 while passing through the axial bore 420 into the distal axial bore 416.

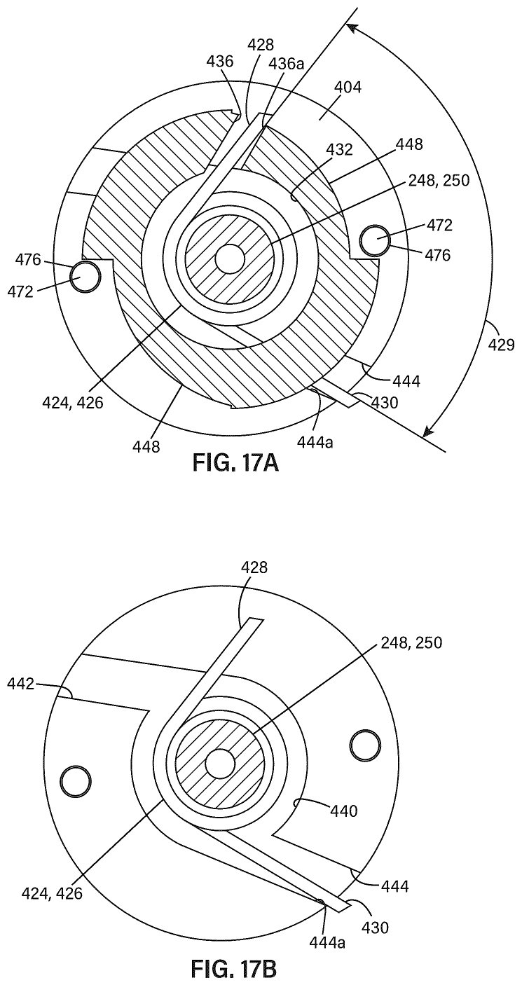

The coil portion 426 of the torsion spring 424 can be arranged in the chamber formed by the aligned recesses 432, 440 with the first end portion 428 extending into the connected lateral slot 436 (as illustrated in FIG. 17A) and the second end portion 430 extending into one of the lateral slots 442, 444 in the first rotatable body 404 (as illustrated in FIG. 17B). In this position, the coil portion 426 is disposed around the portion of the connector shaft 402 extending through the recesses 432, 440 (depicted in FIG. 13B). The central axis of the coil portion 426 is aligned with the longitudinal axis L2 of the torque limiter 400 such that both the rotatable bodies 404, 408 can rotate about the central axis of the coil portion 426.

As illustrated in FIG. 17A, the end portions 430, 428 of the torsion spring 424 can engage surfaces 444a, 436a of the respective receiving slots 444, 436 formed in the rotatable bodies 404, 408. The torsion spring 424 can bias the rotatable bodies 404, 408 into an initial position in which the rotatable bodies 404, 408 rotate together as a single body. The torsion spring 424 is configured to twist in a direction in which the end portions 430, 428 approximate each other when the torque on the actuator driver 248 is within predefined torque limit range. In some cases, the torsion spring 424 can be preloaded, and the torsion spring 424 can start twisting when the torque on the actuator driver 248 exceeds the preload in the torsion spring 424. The preload in the torsion spring 424 can be set as the lower limit of the predetermined range. The upper limit of the predetermined range can be the predetermined torque limit on the actuator driver 248, and the lower limit of the predetermined range can be less than the predetermined torque limit on the actuator driver 248 (e.g., within 10 to 15% of the predetermined torque limit). This means that the torsion spring 424 will start twisting as the actuator driver 248 approaches the predetermined torque limit rather than after the actuator driver 248 reaches or exceeds the predetermined torque limit. In some cases, the predetermined torque limit can be 50 N-mm. The initial angular spacing 429 illustrated in FIG. 17A corresponds to the initial position of the rotatable bodies 404, 408. The angular spacing 429 becomes smaller as the end portions 430, 428 approximate each other during twisting of the torsion spring 424.

As illustrated in FIG. 15, tapered recessed portions 448 can be formed on the outer surface 446 of the second rotatable body 408. In the example, each tapered recessed portion 448 includes a first radial shoulder 452, a second radial shoulder 456 spaced from the first radial shoulder 452 in a circumferential direction of the second rotatable body 408, and a portion 446a of the outer surface 446 between the first and second radial shoulders 452, 456. The outer surface portion 446a can be a curved surface in one example.

The radial projection of the first radial shoulder 452 is greater than the radial projection of the second radial shoulder 456 such that the recessed portion 448 tapers in the radial direction (i.e., deep to shallow) from the first radial shoulder 452 to the second radial shoulder 456. Each tapered recessed portion can extend axially along the entire length of the second rotatable body 408 or partially along the length of the second rotatable body 408. In one example, two tapered recessed portions 448 are formed on the outer surface 446. The tapered recessed portions 448 are angularly spaced from each other about a central axis of the second rotatable body 408, which can be the same as the longitudinal axis L2 of the torque limiter. The angular spacing between the two tapered recessed portions 448 can be such that the two tapered recessed portions are diametrically opposed about the central axis of the second rotatable body 408.

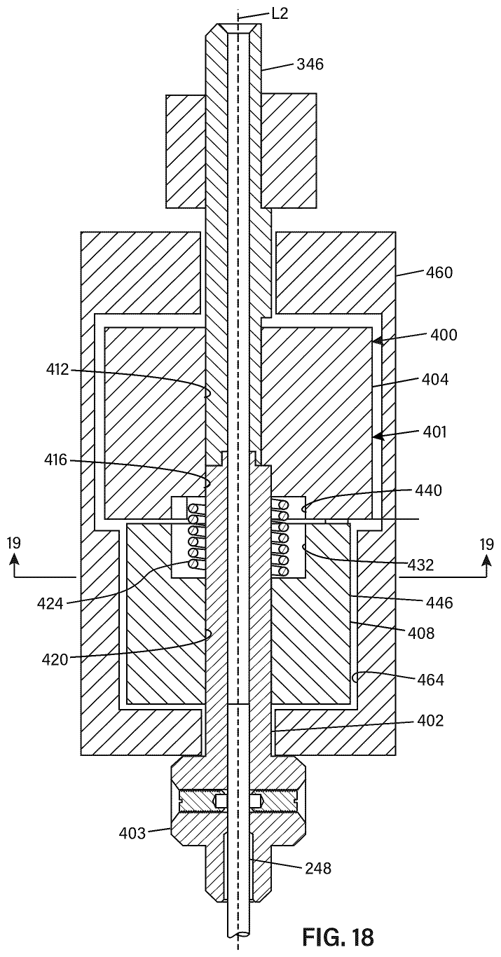

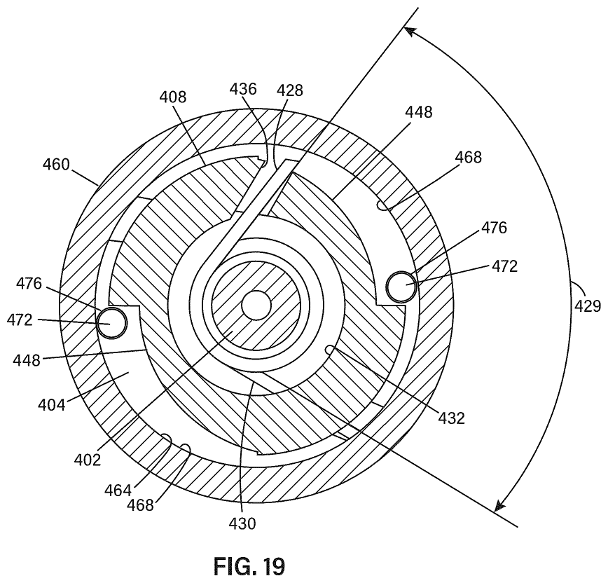

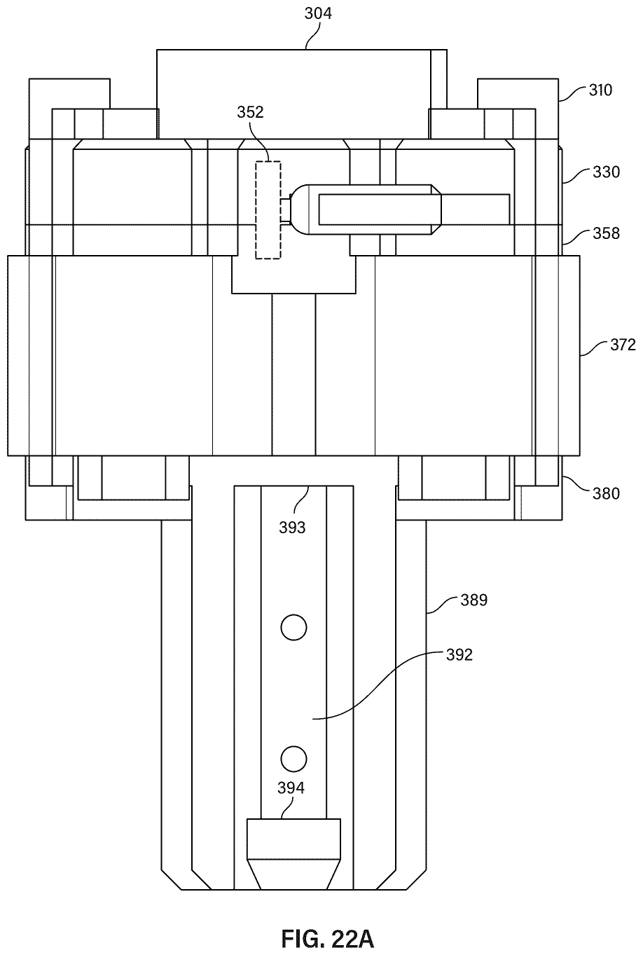

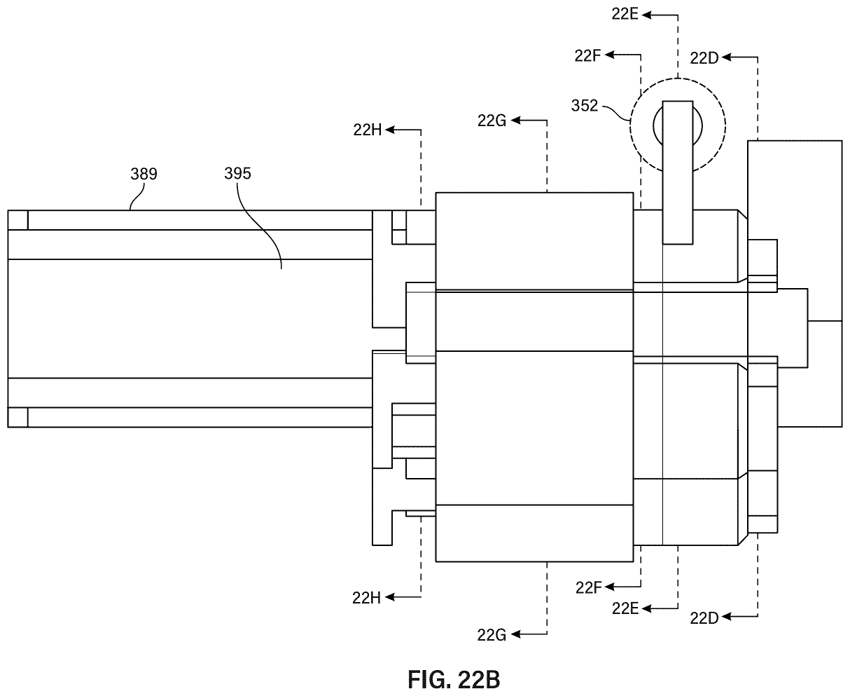

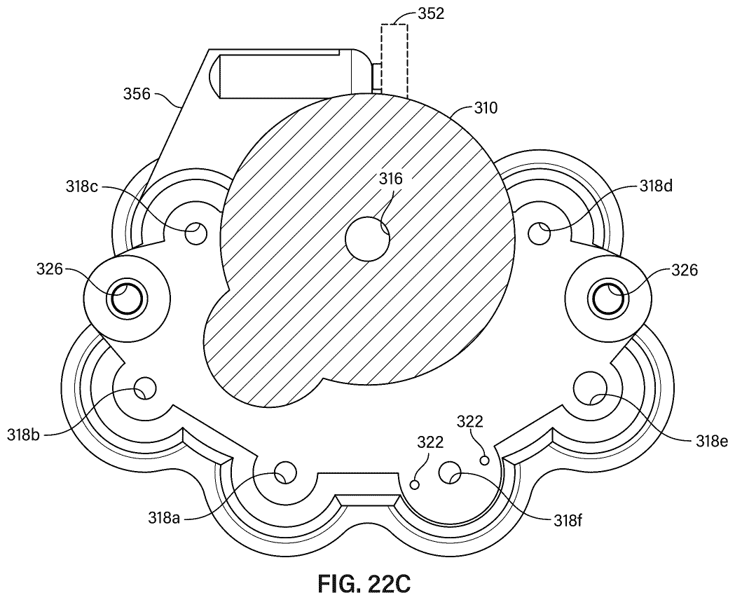

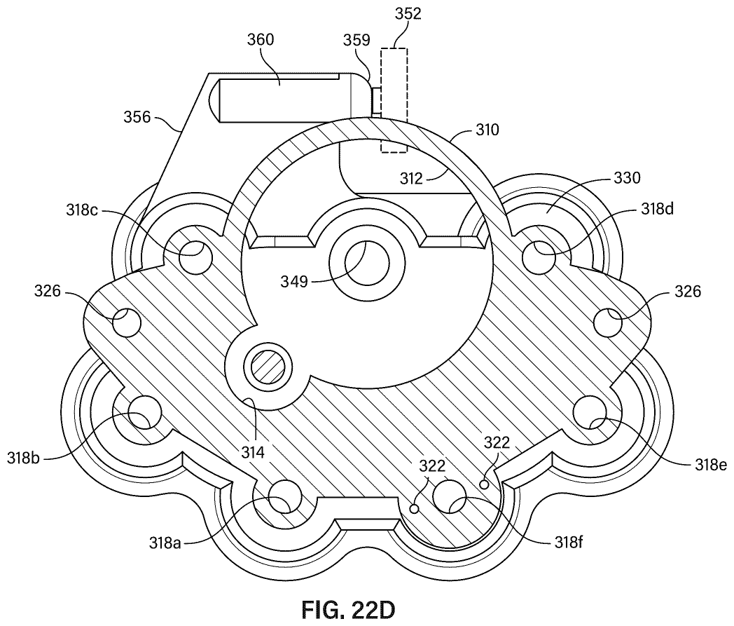

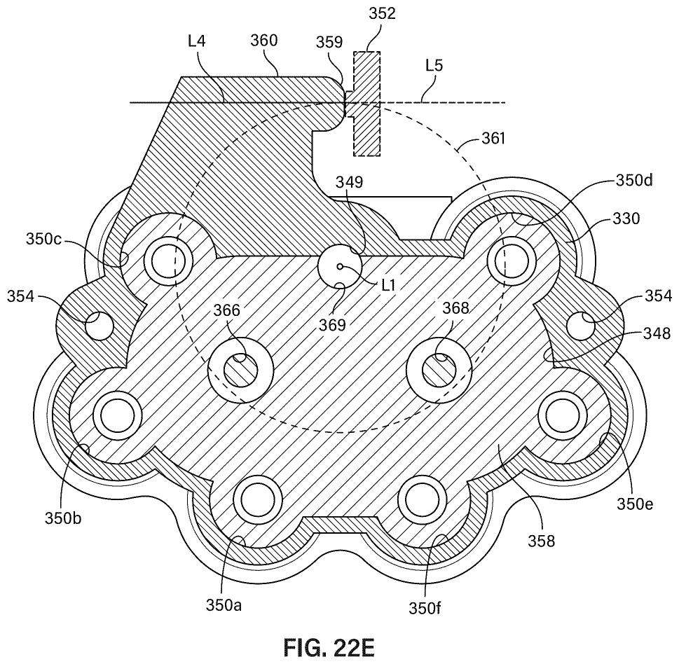

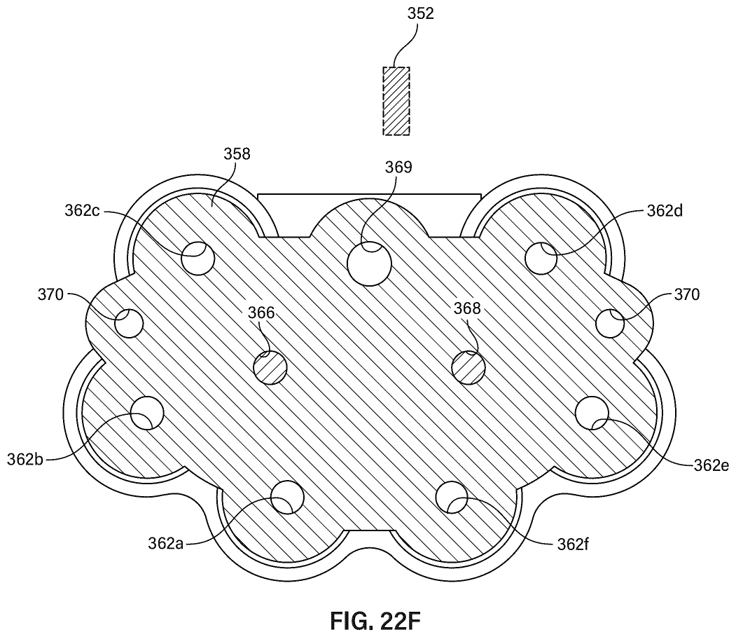

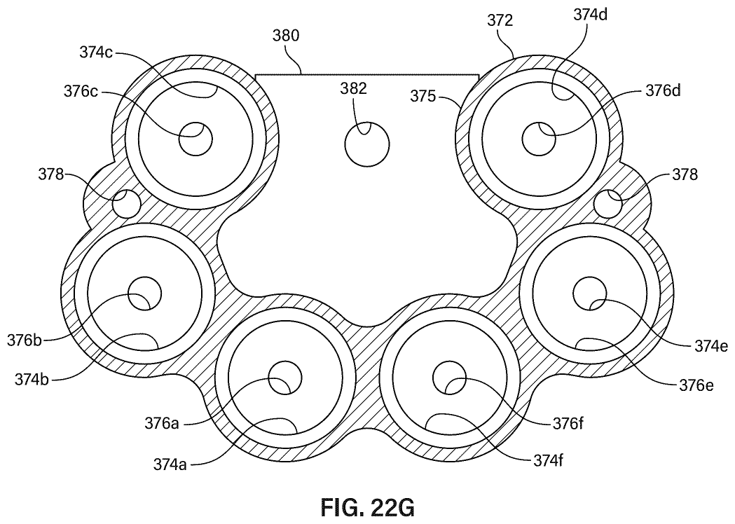

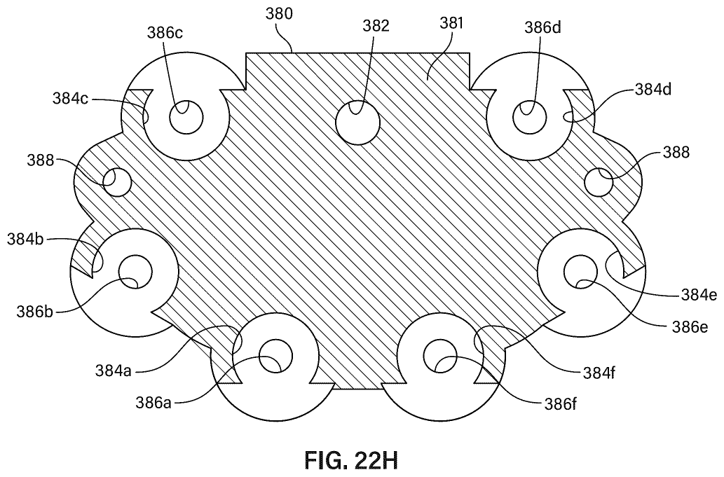

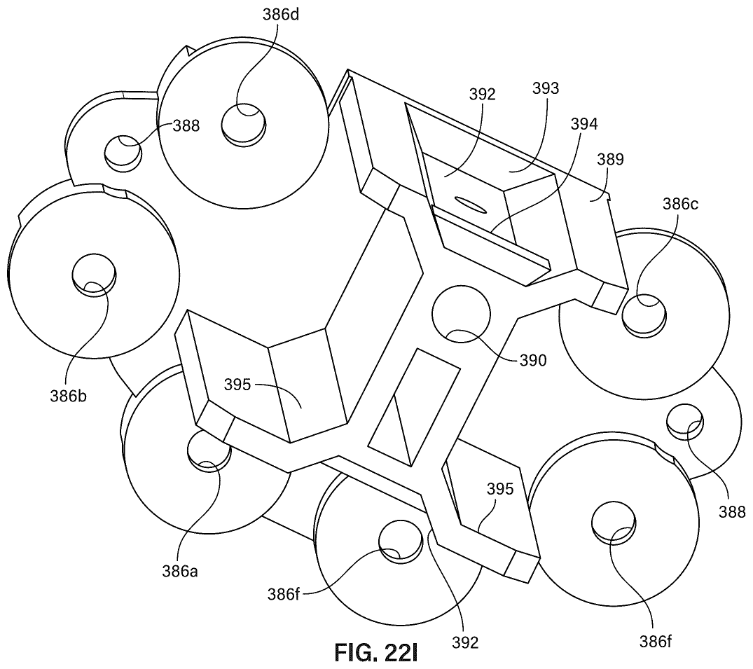

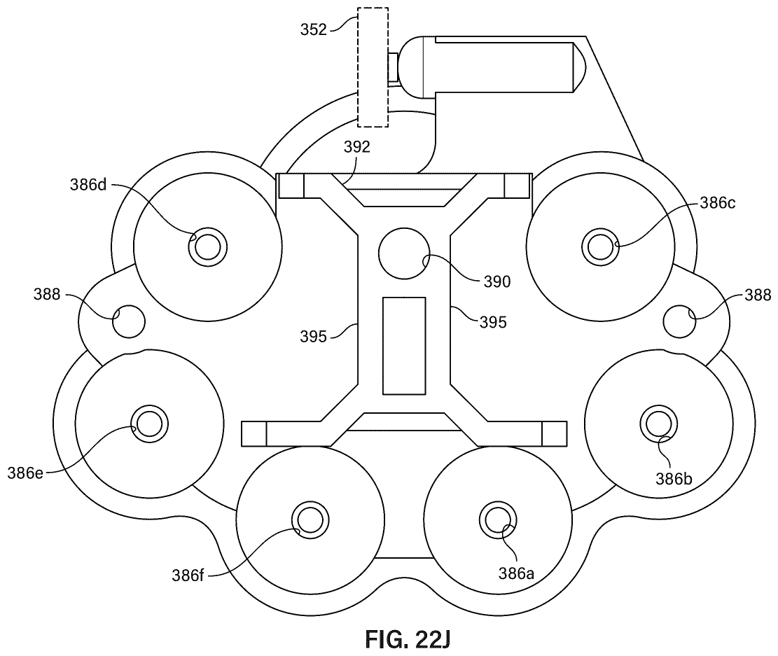

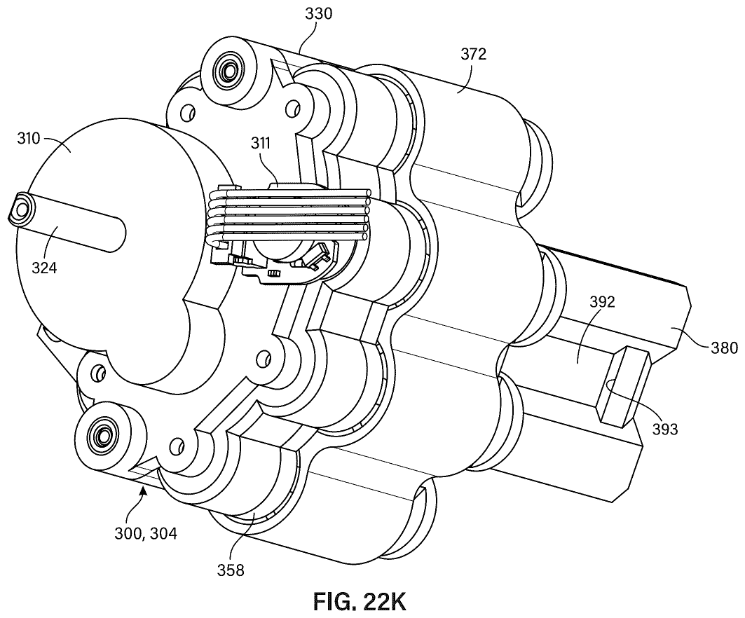

As further illustrated in FIG. 18, the rotatable assembly 401 of the torque limiter 400 can be disposed in a housing 460 such that the outer surface 446 of the second rotatable body 408 is circumscribed by an inner surface 464 of the housing 460. The tapered recessed portions 448 in the outer surface 446 and the inner surface 464 can define circumferentially tapered channels 468 disposed on the periphery of the second rotatable body 408, as illustrated more clearly in FIG. 19. The housing 460 can be a compartment of the gearbox housing 304 (e.g., one of compartments 374a-f illustrated in FIG. 22G) or can be a separate housing that is mounted to the gearbox housing 304.

As shown more clearly in FIG. 19, each channel 468 accommodates a wedge member 472. In one example, the wedge member 472 can be in the form of a longitudinal rod member. In one example, the wedge members 472 are fixedly coupled to the first rotatable body 404 such that the wedge members 472 rotate with the first rotatable body 404. In one example, proximal portions of the wedge members 472 extend into longitudinal holes 476 in the first rotatable body 404 (shown in FIGS. 13A, 16, 17A, 17B, 19). The wedge members 472 can be held in place in the holes 476 using any suitable method (e.g., by friction, welding, gluing, and the like).

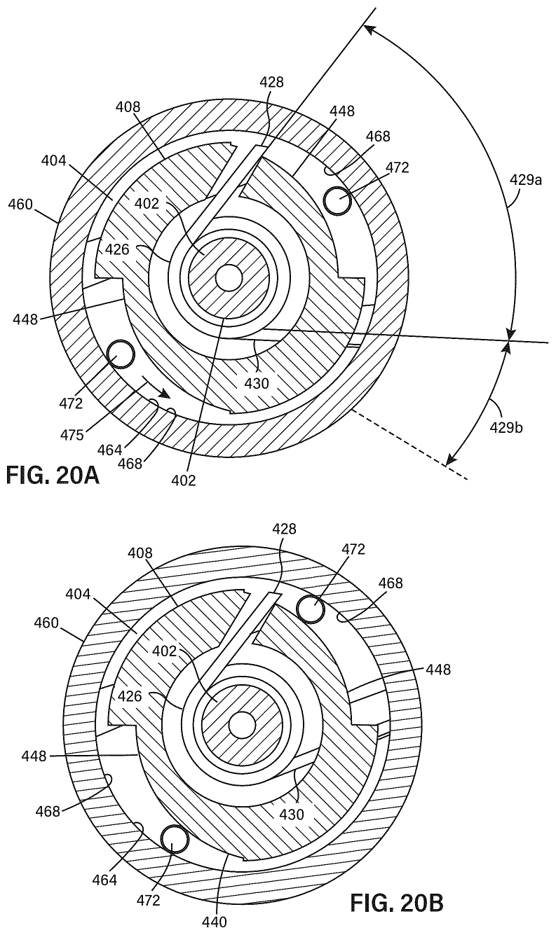

FIGS. 19, 20A, and 20B illustrate operation of the torque limiter 400. The first knob 264 of the handle 204 can be rotated to operate the gear train 308. While the gear train 308 is working, the gear train 308 rotates the output shaft 346. The first rotatable body 404 rotates with the output shaft 346. Rotation of the first rotatable body 404 is translated to rotation of the second rotatable body 408 through the torsion spring 424. As the second rotatable body 408 rotates, the actuator driver 248, which is coupled to the second rotatable body 408 via the connector shaft 402, also rotates. In the state shown in FIG. 19, the torsion spring 424 is in its resting, undeflected state, with an initial angular difference 429 between the end portions 428, 430. In this state, the wedge members 472 are freely accommodated in the wide end of the channels 468, and the first and second rotatable bodies 404, 408 rotate together.

If a torque on the actuator driver 248 reaches a predefined torque limit range set by the size and properties of the torsion spring 424, the coil portion 426 of the torsion spring 424 twists in a manner that approximates the end portions 428, 430 of the torsion spring 424 towards each other. FIG. 20A illustrates an angular spacing 429a between the end portions 428, 430 that is smaller than the initial angular spacing 429 (depicted in FIG. 19) due to the end portions 428, 430 approximating each other (the initial angular spacing 429 shown in FIG. 19 is the sum of the angular spacings 429a, 429b indicated in FIG. 20A). As the torsion spring 424 twists, the first rotatable body 404 rotates relative to the second rotatable body 408 (rather than rotating together), as illustrated in FIG. 20A. As the first rotatable body 404 rotates relative to the second rotatable body 408, the wedge members 472 move along the tapered channels 468 in a direction from the wide end of the channels to the narrow end of the channels, as illustrated by the arrow 475.

The first rotatable body 404 stops rotating when the wedge members 472 are pressed against the narrow end of the channels 468 such that further rotational movement of the wedge members 472 within the tapered channels 468 is not possible due to the interference between the surfaces of the housing 460 and the second rotatable body 408 and the wedge members 472, as illustrated in FIG. 20B. In this state, the second rotatable body 408 also stops rotating. Since all the gears of the gear train 308 are interconnected, once the actuator driver 248 reaches a torque limit that halts rotation of the first and second rotatable bodies 404, 408 and the output gear 344 associated with the actuator driver 248, the movement of the entire gear train 308 stops, preventing rotational movement of all other actuator drivers 248 coupled to the gearbox 300.

In this manner, the torque limiter 400 can help ensure that the actuation members and/or other components of the prosthetic heart valve and/or delivery apparatus are operated within the predetermined torque limits. This can, among other things, reduce or prevent the prosthetic heart valve from being damaged during expansion/contraction and/or prevent the prosthetic heart valve from being overly expanded relative to a native annulus (and/or other native tissue).

The gearbox housing 304 can include various compartments to accommodate the components of the gear train 308 and torque limiter 400, as illustrated in FIGS. 22A-22J.

In one example, as shown in FIG. 22A-22D, the gearbox housing 304 can have a first housing section 310 (which can also be referred to as “first gear housing”) forming a proximal end portion of the gearbox housing. The first housing section 310 can include compartments 312 and 314 to accommodate the input gear 320 (shown in FIGS. 10A-0C) and the transmission gear 328 (shown in FIGS. 10A-10C), respectively. The first housing section 310 can include a hole 316 for passage of a proximal end portion of the input shaft 324 (e.g., to allow coupling of the proximal end portion of the input shaft 324 to the first knob 264 (shown in FIG. 12)). The first housing section 310 can include holes 318a-f for passage of proximal end portions of the output shafts 346a-f (shown in FIGS. 10A-10C). The first housing section 310 can further include fastening holes 326 (shown in FIG. 22C) that can receive fasteners, such as bolts, which can be used to fasten the first housing section 310 to other housing sections of the gearbox housing.

In some cases, the first housing section 310 can include mounting holes 322 for mounting of an encoder about a proximal end portion of one of the output shafts 346a-f. For example, the mounting holes 322 can receive fasteners, such as screws, that are used to attach the encoder to the first housing section 310 and around the respective output shaft. FIG. 22K shows an encoder 311 mounted on one of the output shafts. In one example, the encoder 311 can include a sensing member that can detect the number of rotations of the output shaft. In one example, the encoder can be a magnetic encoder including a magnetic sensor and a magnetic arrangement to generate a magnetic field. The magnetic sensor can detect changes in the magnetic field as the output shaft rotates. Other types of encoders can be used, such as optical encoders.

In one example, as shown in FIGS. 22A-22E, the gearbox housing 304 can have a second housing section 330 (which can also be referred to as “second gear housing”) disposed adjacent to the first housing section 310. The second housing section 330 includes a central opening 348 and compartments 350a-f formed on the periphery of the central opening 348. The central opening 348 can accommodate the driving gears 336, 340 (shown in FIGS. 10A-10C). The compartments 350a-f can accommodate the output gears 344a-f (shown in FIGS. 10A-10C). The compartments 350a-f are longitudinally aligned with the holes 318a-f in the first housing section 310. The second housing section 330 can include fastening holes 354 that can be aligned with the fastening holes 326 in the first housing section 310 to receive fasteners. The second housing section 330 can include an opening 349 that is aligned with the compartment 312 in the first housing section 310. The opening 349 can allow the input shaft 324 to extend through the second housing section 330 when the input gear 320 is mounted in the compartment 312.

In one example, as shown in FIGS. 22A, 22B, 22E, and 22F, the gearbox housing 304 can have a third housing section 358 (which can also be referred to as “shaft support”) disposed adjacent to the second housing section 330 and forming an end wall for the central opening 348 and compartments 350a-f in the second housing section 330. The third housing section 358 can include holes 362a-f (shown in FIG. 22F) to receive the output shafts 346a-f (shown in FIGS. 10A-0C) when the output gears 344a-f (shown in FIGS. 10A-0C) are disposed in the compartments 350a-f of the second housing section 330. The third housing section 358 can include holes 366 and 368 to receive the transmission shaft 332 (shown in FIGS. 10A-10C) and the driving shaft 342 when the driving gears 336, 340 are disposed in the central opening 348 of the second housing section 330. The third housing section 358 can include an opening 369 that is aligned with the opening 349 in the second housing section 330. The opening 369 can allow the input shaft 324 to extend through the third housing section 358 when the input gear 320 is mounted in the compartment 312 of the first housing section 310. The third housing section 358 can include fastening holes 370 that can be aligned with the fastening holes 354 in the second housing section 330 and the fastening holes 326 in the first housing section 310 to receive fasteners.

In one example, as shown in FIGS. 22A, 22B, and 22G, the gearbox housing 304 can have a fourth housing section 372 (which can also be referred to as “torque limiter housing”) disposed adjacent to the third housing section 358. The fourth housing section 372 can include compartments 374a-f arranged in the same pattern as the holes 362a-f in the third housing section 358 and the compartments 350a-f in the second housing section 330. Each of the compartments 374a-f can accommodate a torque limiter 400 (shown in FIG. 18), which can be coupled to a respective output shaft 346a-f extending through a respective hole 362a-f. The fourth housing section 372 include holes 376a-f in end walls of the compartments 374a-f for passage of the connector shafts 402 of the torque limiters 400 outside of the fourth housing 372 when the rotatable assemblies 401 (shown in FIG. 18) of the torque limiters 400 are accommodated within the compartments 374a-f. The fourth housing section 372 can include fastening holes 378 that can be aligned with the fastening holes 370, 354, 326 in the housing sections 358, 330, 310. The compartments 374a-f can be arranged in a pattern to define a channel 375. The input shaft 324 can extend through the channel 375.

In one example, as shown in FIGS. 22A, 22B, 22G, and 22H, the gearbox housing 304 can have a fifth housing section 380 disposed adjacent to the fourth housing section 372. The fifth housing section 380 (which can also be referred to as “release housing”) can form a distal end portion of the gearbox housing 304. The fifth housing section 380 can include a base member 381 forming an end wall for the channel 375. A hole 382 can be formed in the base member 381 to allow the input shaft 324 to pass through the base member 381. The fifth housing section 380 can further include receptacles 384a-f formed in the base member 381 to receive end portions of the torque limiters 400 (shown in FIG. 18) when the rotatable assemblies 401 of the torque limiters 400 are disposed in the compartments 374a-f of the fourth housing section 372. The base member 381 can include openings 386a-f connected to the receptacles 384a-f such that the coupling sections 403 of the connector shafts 402 of the torque limiters 400 can be mounted in the openings 386a-f, or are accessible through the openings 386a-f, when the rotatable assemblies 401 are disposed in the compartments 374a-f in the fourth housing section 372. The fifth housing section 380 can include fastening holes 388 that can be aligned with the fastening holes 378, 370, 354, 326 in the housing sections 372, 358, 330, 310 to receive fasteners.

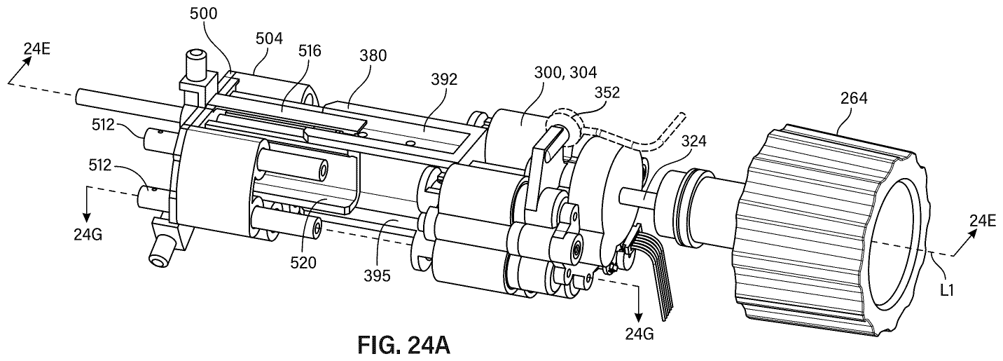

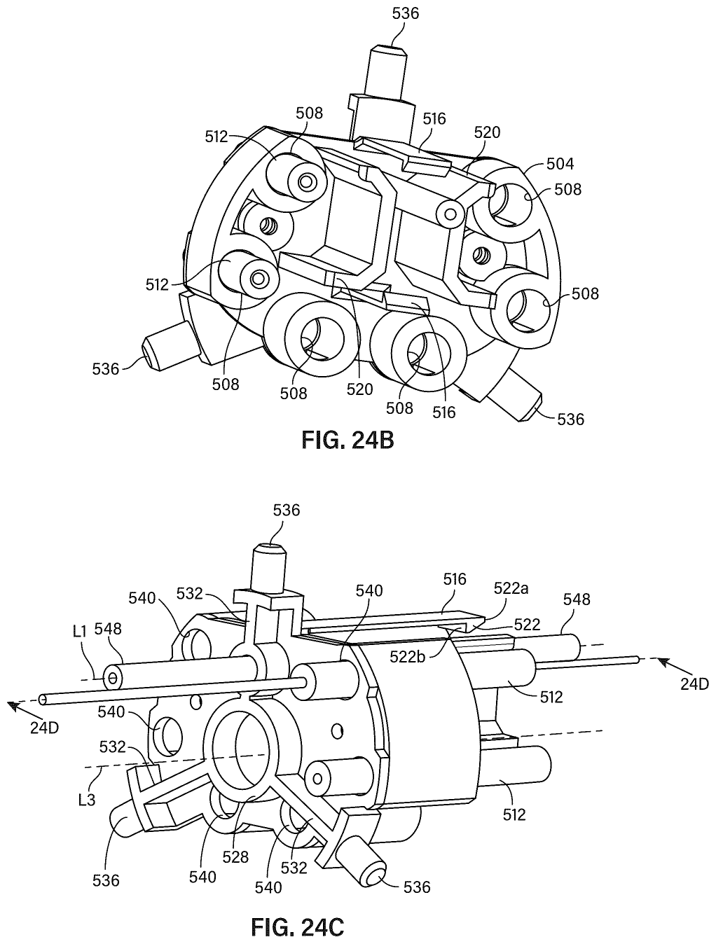

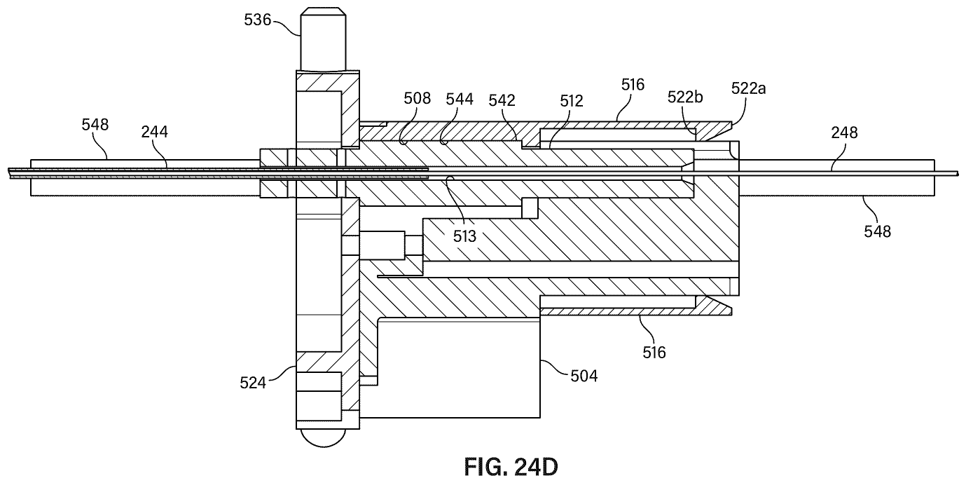

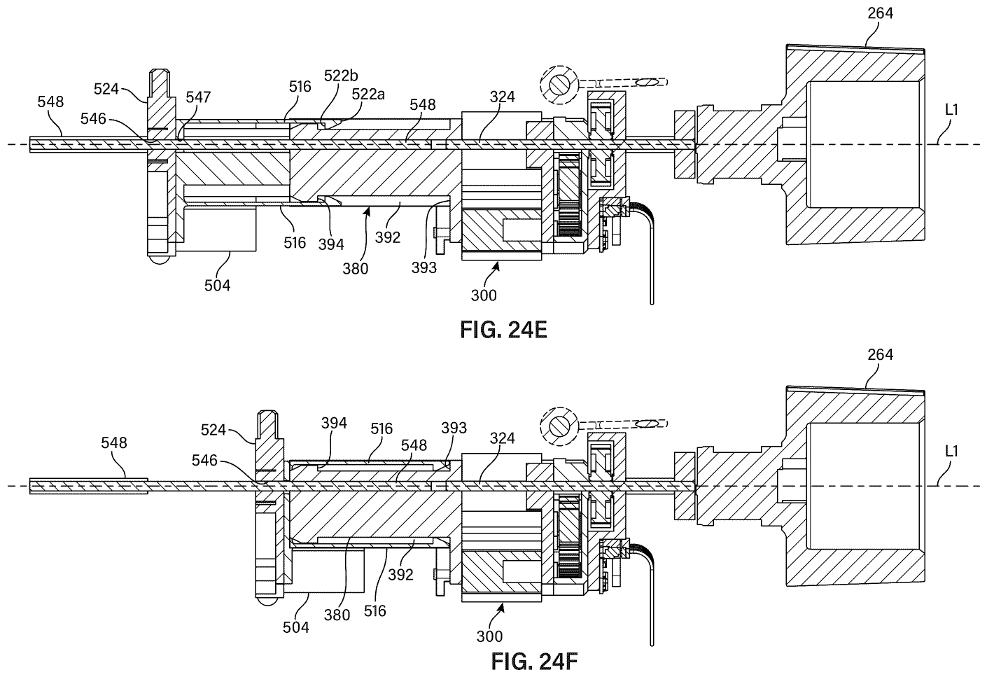

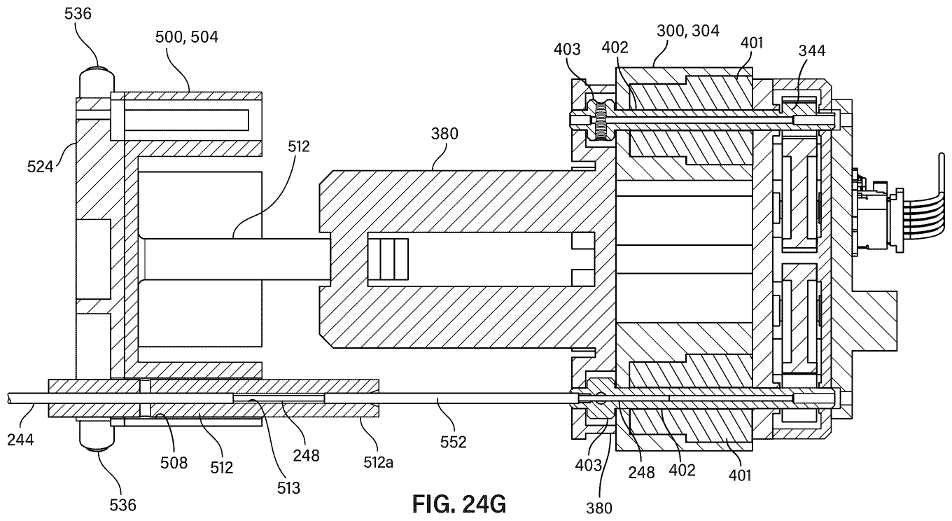

In one example, as shown in FIGS. 22A, 22B, and 22H-22J, the fifth housing section 380 can further include a guide member 389 projecting from the base member 381. The guide member 389 can include a hole 390 aligned with the hole 382 in the plate member 381 to receive an end portion of the input shaft 324. Thus, when the gearbox 300 is fully assembled, the input shaft 324 will extend across all the housing sections 310, 330, 358, 372, and 380 (as shown in FIGS. 24E and 24F). The input shaft 324 defines a longitudinal axis of the gearbox housing, which is also an axis about which the gearbox housing can pivot. The longitudinal axis of the gearbox housing 304 is aligned with the longitudinal axis L1 of the handle 204. A pair of guide slots 392 are formed on opposed surfaces (e.g., top and bottom surfaces) of the guide member 389. The guide slots 392 extend axially in a direction along the longitudinal axis of the gearbox housing. Each guide slot 392 has opposed end walls forming opposed stop surfaces 393, 394. A pair of guide channels 395 are formed on opposed sides of the guide member 389. The guide channels 395 extend axially in a direction along the longitudinal axis of the gearbox housing. As will be further described, the guide slots 392 and guide channels 395 can guide translation of a pull body along the longitudinal axis L1 of the handle.

The various housing sections 310, 330, 358, 372, and 380 of the gearbox housing 304 can be provided as separate members that are fastened together or as integral portions of the gearbox housing 304. In some cases, two or more of the housing sections 310, 330, 358, 372, and 380 can be integrally formed such that the gearbox housing 304 has fewer components to fasten together. In some cases, the gearbox housing 304 can be provided in two halves that can be fastened together. In other cases, the housing sections of the gearbox housing 304 can be attached together using means other than fasteners, e.g., by welding, adhesive, and the like.

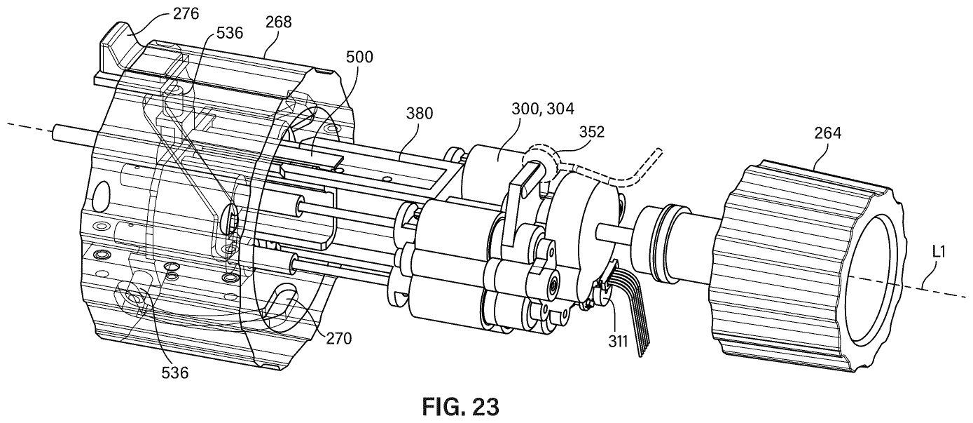

Referring to FIG. 23, the handle 204 can further include a pull body 500 disposed distally to the gearbox 300 and releasably engaged with the fifth housing section/release housing 380 of the gearbox housing 304. The second knob 268 can rotatably engage the pull body 500 such that rotation of the second knob 268 relative to the handle body produces translation of the pull body 500 along the longitudinal axis L1 of the handle. The pull body 500 can be coupled to the outer sleeves 244 of the actuation assemblies 220 such that translation of the pull body 500 along the longitudinal axis L1 of the handle results in axial displacement of the outer sleeves 244 relative to the handle. This axial displacement can be used, for example, to axially displace the outer sleeves 244 relative to the corresponding actuator drivers 248 and thereby release the actuator drivers 248 from the prosthetic heart valve.

Referring to FIGS. 24A-24D, the pull body 500 has an axial axis L3 that is parallel to the longitudinal axis L1 of the handle. The pull body 500 includes a first pull body member 504 having a plurality of elongate sockets 508 that are parallel to the axial axis L3 of the pull body 500. Each of the sockets 508 can receive an actuation tube 512 (only two sockets 508 receiving actuation tubes 512 are illustrated in FIGS. 24A-24C). The number of sockets 508 can match the number of actuation assemblies 220 of the delivery apparatus 200. For example, if the delivery apparatus 200 has six actuation assemblies 220, the pull body 500 can have six sockets 508. The sockets 508 are located on a side of the first pull body member 504 facing the gearbox 300.

The first pull body member 504 can include a pair of slider arms 516 extending in a direction parallel to the longitudinal axis L1 of the handle (and parallel to the axial axis L3 of the pull body) and towards the gearbox 300. With respect to each other, the slider arms 516 are spaced apart in a direction transverse to the longitudinal axis L1 of the handle (e.g., radially) and are in opposed relation. Each guide arm 516 terminates in a hooked end 522 having opposed stop surfaces 522a, 522b, which can be oriented transversely to the longitudinal axis L1/axial axis L3. As illustrated in FIG. 24A, the slider arms 516 can be disposed in the respective guide slots 392 in the release housing 380 of the gearbox housing 304. Each slider arm 516 can move longitudinally within the respective guide slot 392 in a direction parallel to the longitudinal axis L1 of the handle. The opposed stop surfaces 522a, 522b of the hooked end 522 of the slider arm 516 can engage the opposed stop surfaces 393, 394 of the respective guide slot 392 in order to limit travel of the slider arm 516 in the proximal direction or the distal direction. For example, when the stop surface 522a of the hooked end 522 abuts the stop surface 393, further movement of the slider arm 516 in the proximal direction is prevented (as depicted in FIG. 24F). Similarly, when the stop surface 522b of the hooked end 522 abuts the stop surface 394, further movement of the slider arm 516 in the distal direction is prevented (as depicted in FIG. 24E). The distance between the end walls 393, 394 and/or the length of the slider arms 516 can be configured to allow the desired displacement of the pull body 500 and attached outer sleeves 244.

The first pull body member 504 can include a pair of guide beams 520 extending axially in a direction parallel to the longitudinal axis L1 of the handle (and parallel to the axial axis L3 of the pull body 500). Relative to each other, the guide beams 520 are spaced apart in a direction transverse to the longitudinal axis L1 of the handle (e.g., radially) and are in opposed relation. As illustrated in FIG. 24A, the guide beams 520 can be disposed in the respective guide channels 395 in the release housing 380 of the gearbox housing 304. Each guide beam 520 can move longitudinally within the respective guide channel 395 in a direction parallel to the longitudinal axis L1 of the handle as the slider arms 516 move longitudinally within the respective guide slots 392.

The pull body 500 includes a second pull body member 524 disposed adjacent to the first pull body member 504 (e.g., distal in the depicted example). The second pull body member 524 can be attached to the first pull body member 504 by fasteners or other suitable method, such as welding, adhesive, and the like. In some instances, the first pull body member 504 and second pull body member can be formed (e.g., molded) as a single, unitary component. The second pull body member 524 includes a central hub 528 having a longitudinal axis that is aligned with the longitudinal axis L3 of the pull body 500. The second pull body member 524 includes a plurality of radial arms 532 extending from the central hub 528 to a periphery of the pull body 500. The radial arms 532 are angularly spaced about the axial axis L3 of the pull body 500, or angularly offset from each other. Each radial arm 532 comprises a pin 536 that protrudes from the periphery of the pull body 500. The pins 536 are angularly spaced about the axial axis L3 of the pull body 500, or angularly offset from each other, by virtue of the radial arms 532 being angularly spaced about the axial axis L3 of the pull body 500.

The second pull body member 524 has a plurality of openings 540 corresponding in number and position to the plurality of sockets 508 in the first pull body member 504. The actuation tube 512 can thereby extend into the sockets 508 through the openings 540. As further illustrated in FIG. 24D, each actuation tube 512 can have keys 542 (e.g., radial protrusions on the outer diameter of the actuation tube) that are received in slots 544 formed in the sockets 508 to prevent rotation of the actuation tube 512 within the sockets 508 (i.e., the actuation tubes 512 are rotationally fixed relative to the pull body 500).

As illustrated in FIGS. 24E and 24F, the first pull body member 504 and the second pull body member 524 have aligned openings 546, 547 to receive a guide rod 548. The guide rod 548 extends into an opening in the release housing 380 of the gearbox housing 304 and together with the slider arms 516 and guide beams 520 maintain longitudinal alignment of the pull body 500 with the gearbox 300 as the slider arms 516 move within the respective slots 392 in the release housing 380 of the gearbox housing 304.

As illustrated in FIGS. 24D and 24G, the outer sleeve 244 of each actuation assembly 200 extends into the lumen 513 of one of the actuation tubes 512 from the distal end of the actuation tube 512. The outer sleeve 244 is fixedly attached to the respective actuation tube 512 (e.g., using set screws 513) so that the outer sleeve 244 can move when the pull body 500 moves. The actuator rod 248 associated with the outer sleeve 244 extends through the outer sleeve 244 to the release housing 380 of the gearbox housing 304. The actuator rod 248 can be secured to a coupling section 403 (e.g., as depicted in FIG. 18) mounted to the release housing 380 so as to couple the actuator rod 248 to the gearbox 300. In the example, the coupling section 403 is connected to a connector shaft 402, which can be coupled to a torque limiter 401. The torque limiter 401 is coupled to one of the output gears 344 of the gearbox 300. If the handle does not use torque limiters, the connector shaft 402 can be coupled directly to the respective output gear 344.

In one example, as shown in FIG. 24G, the outer sleeve 244 may not extend through the entire lumen 513 of the actuation tube 512. In this example, to provide support to the proximal end portion 512a of the actuation tube 512 that hangs out of the pull body 500, a support extension tube 552 can extend from the coupling section 403 into the portion of the lumen 513 of the actuation tube 512 not occupied by the outer sleeve 244. The support extension tube 552 can be fixedly attached to the coupling section 403 but slidable relative to the actuation tube 512 so that the actuation tube 512 can translate over the support extension tube 552 towards or away from the coupling section 403 as the slider arms 516 move along the slots 392 in the release housing 380 of the gearbox housing 304. In this example, each actuation driver 248 extends through the respective outer sleeve 244, through the portion of the lumen 513 of the actuation tube 512 between the outer sleeve 244 and the support extension tube 522, through the support extension tube 552, through the coupling section 403, into the connector shaft 402 (as depicted in FIG. 24G).

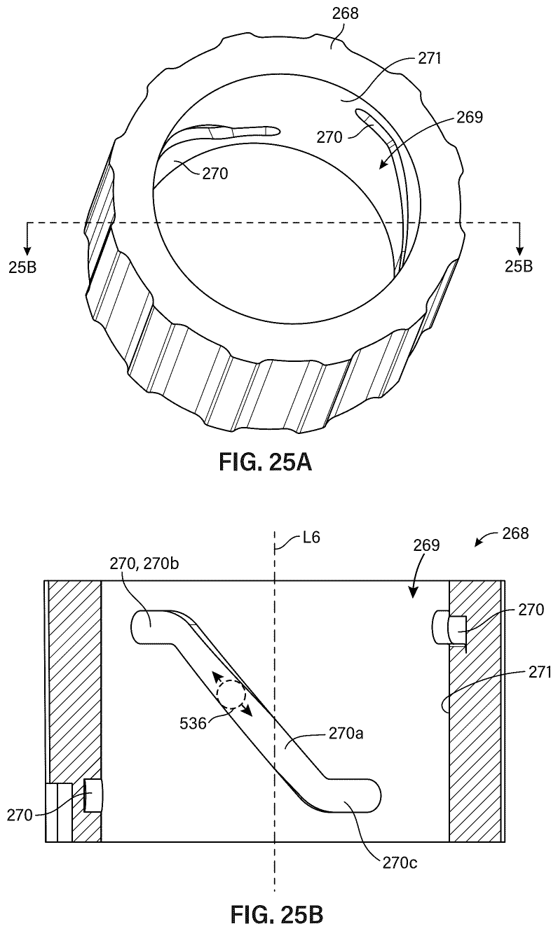

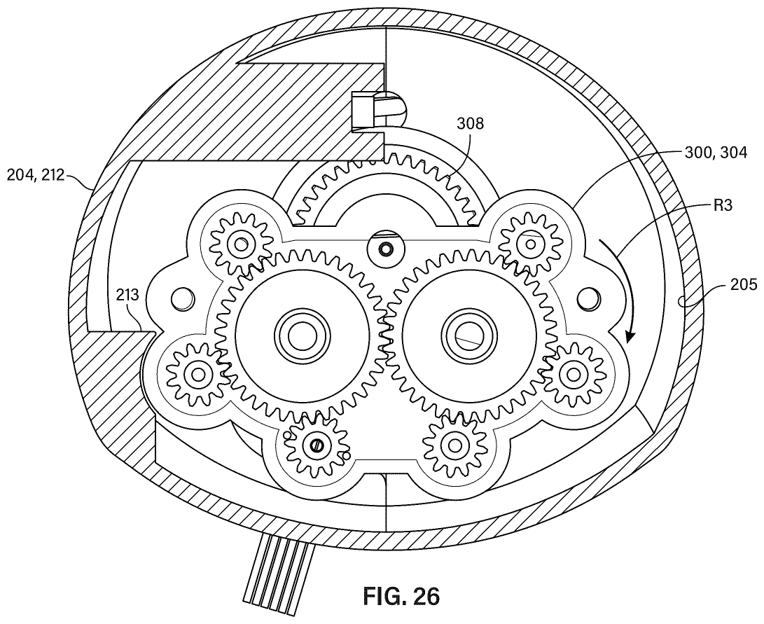

Referring to FIGS. 25A and 25B, the second knob 268 can have an inner surface 271 forming a cylindrical lumen 269. In the example, the cylindrical lumen 269 defines a central axis L6, which is oriented parallel to the longitudinal axis L1 of the handle when the second knob 268 is mounted on the handle body (e.g., as depicted in FIGS. 8 and 23). Cam slots 270 are formed in the inner surface 271 of the second knob 268 that defines the lumen 269). The number of cam slots 270 can match the number of pins 536 of the pull body 500. The cam slots 270 can be rotationally offset from each other about the central axis L6 of the second knob 268 (e.g., for three cam slots 270, the center points of the cam slots 270 can be 120 degrees apart) such that when the second knob 268 is disposed around the pull body 500 (as illustrated in FIG. 23), each pin 536 can extend into a corresponding cam slot 270.

Referring to FIG. 25B, each cam slot 270 can have an angled portion 270a and lateral portions 270b, 270c at the opposite ends of the angled slot portion 270a. The angled slot portion 270a can extend along a path that is angled relative to the central axis L6. For example, the angled slot portion 270a can extend along a helical path. In one example, the path along which the angled slot portion 270a extends, or the inclination angle of the angle slot portion 270a relative to the central axis L6, can be configured such that movement of the pin 536 within and along the angled slot portion 270a produces movement of the pull body 500 in a direction parallel to the longitudinal axis L1 of the handle (or the central axis L6 of the second knob 268).

Each pin 536 can slide into the horizontal slot portions 270b, 270c of the respective cam slot 270 upon reaching the end of a movement of the pull body 500 in a proximal direction or in a distal direction. As shown in FIGS. 4A, 8, and 23, the handle 204 can include a safety knob 276 that can slide into a slot in the second knob 268 to prevent the second knob 268 from being rotated relative to the handle body. The safety knob 268 can be used to restrict rotation of the second knob 268 relative to the body of the handle 204 and thereby prevent or reduce the likelihood of accidental release of the actuation assemblies 220 from the prosthetic heart valve 100. In one example, the second knob 268 can be configured such that the safety knob 276 can slide into the receiving slot in the second knob 268 when the pins 536 are in either of the horizontal slot portions 270b, 270c. In some examples, the safety knob (which can also be called “a safety switch”) can be biased (e.g., with a biasing member such as a spring) toward the engaged/locked position with the second knob 268. When the safety knob 276 is removed from the receiving slot in the second knob 268 (e.g., by sliding the safety knob 276 distally), it will be possible to rotate the second knob 268 such that the pins 536 can be displaced into the angled slot portions 270a.

When the second knob 268 is rotated relative to the handle body, the pins 536 slide along the cam slots 270. As the pins 536 slide along the angled slot portions 270a of the cam slots 270, the pull body 500 is translated along the longitudinal axis L1 of the handle. FIG. 7C shows the outer sleeves 244 engaged with the frame 104 of the prosthetic heart valve 100 and covering the flexible elongated elements 254 (shown in FIG. 7B) of the actuator drivers 248. To release the actuation assemblies 220 from the prosthetic heart valve 100 (e.g., after radially expanding the prosthetic heart valve 100 at the implantation location), the outer sleeves 244 need to be released from the frame 104 and also from covering the flexible elongated elements 254 of the actuator drivers 248. Both actions can be achieved by axially displacing the outer sleeves 244 relative to the handle. For example, the second knob 268 can be rotated in a direction to move the pull body 500 proximally (i.e., towards the gearbox 300). Since the outer sleeves 244 are attached to the pull body 500, the outer sleeves 244 are axially displaced in a direction along the longitudinal axis L1 of the handle and towards the proximal end of the handle. The axial displacement of the outer sleeves 244 can retract the outer sleeves 244 from the frame 104 and from the flexible elongated elements 254, allowing the flexible elongated elements 254 (shown in FIGS. 7A-7B) of the actuator drivers 248 to be released from the actuator heads 176 of the prosthetic heart valve 100.

During expansion of the prosthetic heart valve 100, rotational movement of the actuator drivers 248 by operation of the gearbox 300 applies a torque to the prosthetic heart valve 100 that tends to rotate the prosthetic heart valve about the longitudinal axis L of the prosthetic heart valve. Since the outer sleeves 244 are engaged with the frame 104 of the prosthetic heart valve 100, the outer sleeves 244 tend to rotate around the longitudinal axis L of the prosthetic heart valve 100. Since the pull body 500 is coupled to the outer sleeves 244, the pull body 500 likewise tends to rotate with the outer sleeves 244.

The gearbox 300 can pivot about the longitudinal axis L1 of the handle, which is aligned with the axial axis of the input shaft 324 and the axial axis of the guide rod 548. Thus, rotation of the pull body 500 during expansion of the prosthetic heart valve 100 can result in pivoting of the gearbox 300 about the longitudinal axis L1 of the handle 204. In some examples, the handle 204 includes a mechanism to limit pivoting of the gearbox 300 at least during expansion of the prosthetic heart valve 100. In one example, the mechanism can include a stop member that engages the gearbox housing 304 when the gearbox housing 304 is in a predetermined rotational position relative to the body of the handle 204.

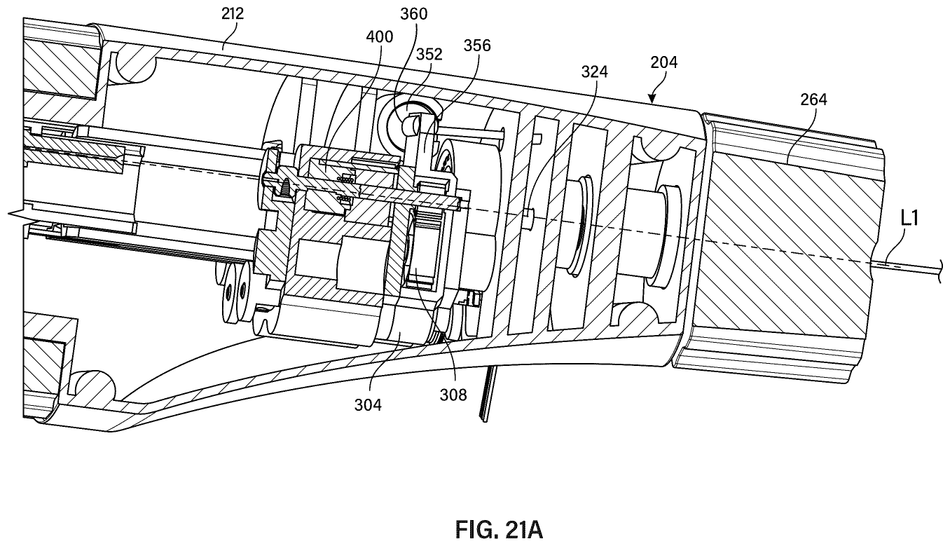

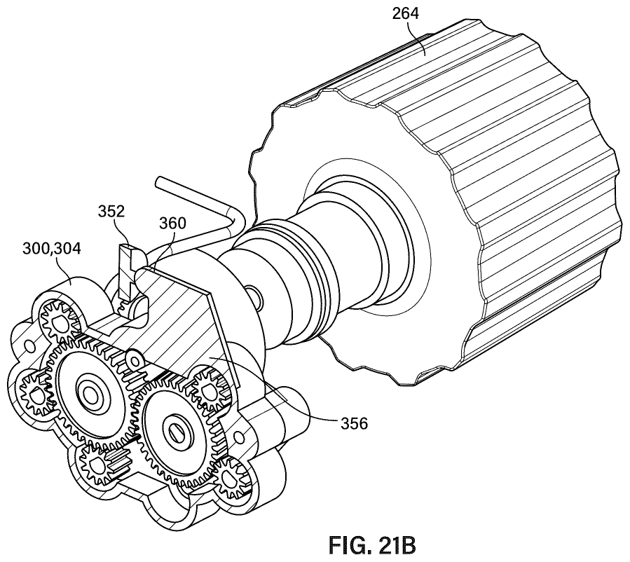

Referring to FIGS. 21A, 21B, 22C-22E, the gearbox housing 304 can have an extension arm 356 projecting from an outer surface of the gearbox housing 304. When the gearbox housing 304 is positioned within the cavity 205 of the handle 204, the extension arm 356 extends into a portion of the cavity 205 surrounding the gearbox housing 304 (as shown in FIG. 21A). In one example, the extension arm 356 can be a flat member lying in a plane transverse to the longitudinal axis L1 of the handle. A protrusion member 360 can be attached to or integrally formed with the extension arm 356. The protrusion member 360 can be in the form of a rod or pin. The protrusion member 360 can have a rounded end 359 for contact with a stop member. The protrusion member 360 can be oriented in a direction transverse to the longitudinal axis L1 of the handle. The extension arm 356 can position the protrusion member 360 such that an axial axis L4 (depicted in FIG. 22E) of the protrusion member 360 is tangential to a circular path 361 centered around the pivoting axis of the gearbox 300 (or longitudinal axis L1 of the handle). This could also be described as the protrusion member 360 being radially outward of the pivoting axis of the gearbox 300. Thus, the protrusion member 360 moves along the circular path 361 as the gearbox 300 pivots.

The extension arm 356 is shown as an integral part of the housing section 330 of the gearbox housing 304. However, the extension arm 356 could be an integral part of any of the other housing sections of the gearbox housing in other examples. Also, the extension arm 356 is shown at the top of the housing section 330. However, it could be located elsewhere on the housing section 330 provided that it positions the protrusion member 360 along the circular path 361. Alternatively, the circular path can be larger or smaller than the circular path 361 so long as it is coaxial with the longitudinal axis L1 of the handle.

A stop member 352 can be mounted to an inner surface of the proximal body portion 212 of the handle 204 (as depicted in FIG. 21A) such that the protrusion member 360 can contact the stop member 352 as the protrusion member 360 moves along the circular path 361. The stop member 352 can be positioned such that an axial axis L5 (shown in FIG. 22E) of the stop member 352 is also tangential to the circular path 361. Thus, as the protrusion member 360 moves along the circular path 361, the protrusion member 360 will encounter the stop member 352. In one example, the stop member 352 can be positioned such that when the first knob 264 (shown in FIG. 21A) is rotated in a direction to expand the prosthetic heart valve (e.g., in the clockwise direction when viewing from the proximal end of the handle), the stop member 352 acts to limit the pivoting of the gearbox 300.

In one example, the first knob 264 can be rotated in a direction to expand the prosthetic heart valve 100 (e.g., in the clockwise direction when viewing from the proximal end of the handle). As the prosthetic heart valve 100 is expanded, if the protrusion member 360 is not yet in contact with the stop member 352, the entire gearbox 300 can pivot about the longitudinal axis L1 of the handle (which is the same as the axial axis of the input shaft 324 as depicted in FIG. 21A) in a direction towards the stop member 352. As the gearbox 300 pivots, the protrusion member 360 moves along the circular path 361 until the protrusion member 360 encounters the stop member 352, which then prevents further pivoting of the gearbox 300 in the same direction. While the protrusion member 360 is in contact with the stop member 352, the gear train 308 can still be operated through rotation of the first knob 264 and input shaft 324.

In one example, the stop member 352 can be a load cell (or force sensor) such that when the protrusion member 360 is in contact with the stop member 352 during expansion of the prosthetic heart valve (as depicted more clearly in FIG. 21B; the stop member 352 is shown in FIG. 21B without the handle body to which it is coupled for simplicity of illustration), any load applied to the stop member 352 by the protrusion member 360 can be measured. This measured load can be used to determine the torque applied to the prosthetic heart valve 100 during expansion of the valve. For example, the measured load can be multiplied by the moment arm as defined by the extension arm 356. Thus, the stop member 352 implemented with a load cell can perform the function of limiting pivoting of the gearbox 300 and measuring torque applied to the prosthetic heart valve 100. Load cells can be provided in dimensions that are significantly smaller than those of conventional torque meters, allowing a more compact handle design.

As the first knob 264 is rotated in a direction that compresses the prosthetic heart valve 100 (e.g., in the counterclockwise direction when viewing from the proximal end of the handle), the protrusion member 360 is spaced away from the stop member 352. As such, the stop member 352 does not act to limit pivoting of the gearbox 300 and does not measure torque when the prosthetic heart valve 100 is being compressed. In some cases, the handle body can act to limit pivoting of the gearbox 300 during compression of the prosthetic heart valve 100. For example, as illustrated in FIG. 26, the proximal body portion 212 of the handle 204 can include an inner protrusion 213 that engages the gearbox 300 when the gearbox 300 pivots in a direction R3 corresponding to compression of the prosthetic heart valve (e.g., the counterclockwise direction when viewed from the proximal end of the handle or the clockwise direction when viewed from the distal end of the handle). In some cases, the gearbox 300 can be provided with a second extension arm and protrusion member, and a second load cell (or a multi-directional load sensor) can be mounted on the handle body. The protrusion member on the second extension arm can be arranged to contact the second load cell to measure the torque applied to the prosthetic heart valve 100 while compressing the prosthetic heart valve.

Referring to FIGS. 1-26, the prosthetic heart valve 100 can be placed in a radially compressed configuration, and the actuation assemblies 220 of the delivery apparatus 200 can be releasably coupled to the actuators 168 of the prosthetic heart valve 100. The delivery apparatus 200 and the prosthetic heart valve 100 can be advanced over a guidewire through the vasculature of a patient to a selected implantation location (e.g., the native aortic annulus). For example, when implanting the prosthetic heart valve 100 within the native aortic valve, the delivery apparatus 200 and the prosthetic heart valve 100 can be inserted into and through a femoral artery, and through the aorta to the native aortic valve. The prosthetic heart valve 100 can then be deployed at the implantation location.

In one example, the prosthetic heart valve 100 is enclosed in a delivery capsule 226 prior to insertion into the patient's vasculature. In this case, the third knob 272 can be operated to retract the delivery capsule 226 and expose the prosthetic heart valve 100. To deploy the prosthetic heart valve 100, the physician can turn the first knob 264 to rotate the set of first actuator drivers (e.g., 248a, 248b, 248c) in a first direction and the set of second actuator drivers (e.g., 248d, 248e, 248f) in a second direction, corresponding to counter-rotation of the first and second sets of the actuators of the prosthetic heart valve 100 in a direction that radially expands the prosthetic heart valve 100.

During the valve expansion, the torque exerted on the native anatomy can be measured via the stop member/load cell 352 in the handle 204. During the valve expansion, torque limiter(s) 400 can stop the gearbox 300 if respective actuator driver(s) 248 become overloaded. After the prosthetic heart valve 100 has been expanded to the working diameter by rotation of the actuators, the actuation assemblies 220 can be released from the prosthetic heart valve 100. To release the actuation assemblies 220, the pull body 500 can be translated proximally along the longitudinal axis L1 of the handle 204 (e.g., by rotating the second knob 268) so as the retract the outer sleeves 244 from the frame 104 of the prosthetic heart valve 100 and the flexible elongated elements 254 of the actuator drivers 248. The freed flexible elongated elements 254 can be removed from the actuator heads 176 of the prosthetic heart valve 100, allowing the delivery apparatus to be withdrawn from the body.

Any of the systems, devices, apparatuses, etc. herein can be sterilized (for example, with heat, radiation, and/or chemicals, etc.) to ensure they are safe for use with patients, and any of the methods herein can include sterilization of the associated system, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### ADDITIONAL EXAMPLES

Additional examples based on principles described herein are enumerated below. Further examples falling within the scope of the subject can be configured by, for example, taking one feature of an example in isolation, taking more than one feature of an example in combination, or combining one or more features of one example with one or more features of one or more other examples.

Example 1: A delivery apparatus for a prosthetic heart valve comprising a handle body having a cavity and defining a longitudinal axis; a pull body disposed within the cavity and movable within the cavity and along the longitudinal axis; an actuation assembly comprising a sleeve member having an end portion disposed within the cavity and coupled to the pull body such that movement of the pull body along the longitudinal axis axially displaces the sleeve member relative to the handle body; and a knob rotatably mounted on the handle body and operatively coupled to the pull body such that rotation of the knob relative to the handle body moves the pull body within the cavity and along the longitudinal axis.

Example 2: The delivery apparatus of any example herein, particularly Example 2, wherein the pull body defines an axial axis that is oriented parallel to the longitudinal axis and includes at least one pin member disposed radially to the axial axis and protruding from a periphery of the pull body.

Example 3: The delivery apparatus of any example herein, particularly Example 2, wherein the knob includes a cam slot, and that the knob is disposed around the periphery of the pull body such that the pin member extends into the cam slot.

Example 4: The delivery apparatus of any example herein, particularly Example 3, wherein the knob includes an inner surface forming a cylindrical lumen, and that the cylindrical lumen defines a central axis that is oriented parallel to the longitudinal axis, and that the cam slot is formed in the inner surface and includes an angled slot portion.

Example 5: The delivery apparatus of any example herein, particularly Example 4, wherein the angled slot portion is configured such that the pull body moves in a direction parallel to the longitudinal axis as the pin member moves along the angled slot portion.

Example 6: The delivery apparatus of any example herein, particularly Example 1, wherein the pull body defines an axial axis oriented parallel to the longitudinal axis and includes a plurality of pin members disposed radially to the axial axis, and that the pin members are rotationally offset about the axial axis and protrude from a periphery of the pull body.

Example 7: The delivery apparatus of any example herein, particularly Example 6, wherein the knob includes an inner surface forming a cylindrical lumen having a central axis, the knob includes a plurality of cam slots formed in the inner surface and rotationally offset from each other about the central axis, and the knob is disposed around the periphery of the pull body such that each of the pin members extends into one of the cam slots.

Example 8: The delivery apparatus of any example herein, particularly Example 7, wherein each of the cam slots includes an angled slot portion extending along a path that is angled relative to the central axis.

Example 9: The delivery apparatus of any example herein, particularly Example 8, and further specifies that the central axis is oriented parallel to the longitudinal axis and each path is configured such that the pull body moves in a direction parallel to the longitudinal axis as the pin members move along the angled slot portions of the respective cam slots.

Example 10: A delivery apparatus for a prosthetic heart valve comprises a handle body having a first end, a second end, a cavity between the first end and the second end, and a longitudinal axis extending through the first end, the cavity, and the second end; an actuator driver having a first driver end portion disposed within the cavity, a second driver end portion disposed outside the cavity, and a first axial axis extending through the first driver end portion and the second driver end portion; a sleeve member operatively coupled to the actuator driver, the sleeve member having a first sleeve end portion disposed within the cavity, a second sleeve end portion disposed outside the cavity, and a second axial axis extending through the first sleeve end portion and the second sleeve end portion, the second axial axis parallel to the first axial axis; and a pull body disposed within the cavity and coupled to the first sleeve end portion, the pull body movable along the longitudinal axis such that movement of the pull body axially displaces the sleeve member relative to the handle body.

Example 11: The delivery apparatus of any example herein, particularly Example 10, wherein the sleeve member is disposed around the actuator driver.

Example 12: The delivery apparatus of any example herein, particularly Example 10 or Example 11, wherein the sleeve member is coaxial with the actuator driver.

Example 13: The delivery apparatus of any example herein, particularly any one of Examples 10-12, wherein the second driver end portion includes an engagement member to releasably couple the actuator driver to an actuator of the prosthetic heart valve.

Example 14: The delivery apparatus of any example herein, particularly Example 13, wherein the sleeve member is extendible over the engagement member and retractable from the engagement member by movement of the pull body along the longitudinal axis.

Example 15: The delivery apparatus of any example herein, particularly Example 13 or 14, wherein the engagement member includes a central protrusion and one or more flexible elongated elements disposed about the central protrusion.

Example 16: The delivery apparatus of any example herein, particularly any one of Examples 10-15, further comprises a gearbox disposed within the cavity and coupled to the actuator driver for rotation of the actuator driver.

Example 17: The delivery apparatus of any example herein, particularly of Example 16, wherein the gearbox includes a gearbox housing enclosing a gear train, and that the pull body is movably coupled to the gearbox housing.

Example 18: The delivery apparatus of any example herein, particularly Example 17, further comprising a knob coupled to the pull body, wherein rotation of the knob relative to the handle body moves the pull body along the longitudinal axis.

Example 19: The delivery apparatus of any example herein, particularly Example 18, wherein the pull body includes a first pull body portion, a second pull body portion, and a third axial axis extending through a center of the first pull body portion and a center of the second pull body portion, and that the third axial axis is parallel to the longitudinal axis.

Example 20: The delivery apparatus of any example herein, particularly Example 19, wherein the first pull body portion includes a pin member disposed radially to the third axial axis and positioned on a periphery of the pull body, and that the knob includes a cam slot, and that the knob is disposed around the pull body such that the pin member extends into the cam slot.

Example 21: The delivery apparatus of any example herein, particularly Example 20, wherein the knob includes an inner surface forming a cylindrical lumen, and that the cylindrical lumen defines a fourth axial axis that is oriented parallel to the longitudinal axis, and that the cam slot comprises an angled slot portion formed in the inner surface and extending along a path that is angled relative to the fourth axial axis.

Example 22: The delivery apparatus of any example herein, particularly Example 20 or 21, wherein the first pull body portion comprises a central hub and a radial arm projecting from the central hub, and that the pin member is formed at an end of the radial arm.

Example 23: The delivery apparatus of any example herein, particularly Examples 19 to 22, wherein the second pull body portion includes a socket extending in a direction parallel to the third axial axis, and that the first pull body portion comprises an opening forming an extension of the socket.

Example 24: The delivery apparatus of any example herein, particularly Example 23, further comprises an actuation tube extending through the opening and socket, and further specifies that the sleeve member is rigidly coupled to the actuation tube, and that the actuation tube is rotationally fixed relative to the pull body.

Example 25: The delivery apparatus of any example herein, particularly Example 19, wherein the first pull body includes a plurality of pin members disposed radially to the third axial axis and positioned on a periphery of the pull body, and that the pin members are angularly offset from each other.

Example 26: The delivery apparatus of any example herein, particularly Example 25, wherein the knob includes a plurality of cam slots, and that the knob is disposed around the pull body such that each of the pin members is received in one of the cam slots.

Example 27: The delivery apparatus of any example herein, particularly Example 26, wherein the knob includes an inner surface forming a cylindrical lumen, and that the cylindrical lumen defines a fourth axial axis, and that each of the cam slots comprises an angled slot portion formed in the inner surface and extending along a path that is angled relative to the fourth axial axis.

Example 28: The delivery apparatus of any example herein, particularly any one of Examples 25-27, wherein the first pull body portion includes a central hub and a plurality of radial arms projecting from the central hub and angularly offset from each other, and that each of the pin members is formed at an end of one of the radial arms.

Example 29: The delivery apparatus of any example herein, particularly any one of Examples 25-28, further comprising additional actuator drivers and additional sleeve members, each additional sleeve member operatively coupled to one of the additional actuator drivers.

Example 30: The delivery apparatus of any example herein, particularly Example 29, wherein the second pull body portion includes a plurality of sockets extending in a direction parallel to the third axial axis, and that the first pull body portion includes a plurality of openings, each of the openings forming an extension of one of the sockets.

Example 31: The delivery apparatus of any example herein, particularly Example 30, further comprising a plurality of actuation tubes, each of the actuation tubes extending through one of the sockets and the corresponding opening, and that each of the sleeve member and additional sleeve members is rigidly coupled to one of the actuation tubes, and that each of the actuation tubes is rotationally fixed relative to the pull body.

Example 32: The delivery apparatus of any example herein, particularly Example 31, wherein for each of the sockets and the actuation tube extending through the socket, one of the sockets and a respective actuation tube includes a key, and the other of the sockets and respective actuation tube includes a slot, and that the key and the slot cooperate to prevent rotation of the actuation tube within the socket.

Example 33: The delivery apparatus of any example herein, particularly any one of Examples 19-32, wherein the second pull body portion comprises a pair of slider arms projecting in a direction parallel to the third axial axis and spaced from each other in a direction transverse to the third axial axis.

Example 34: The delivery apparatus of any example herein, particularly Example 33, wherein each slider arm comprises an end portion having a pair of opposed surfaces oriented transversely to the third axial axis.

Example 35: The delivery apparatus of any example herein, particularly any one of Examples 33-34, wherein an end portion of the gearbox housing includes a pair of slots extending in a direction along the longitudinal axis, and that the pair of slider arms extend into the pair of slots and are movable along the pair of slots in response to movement of the pull body along the longitudinal axis.

Example 36: The delivery apparatus of any example herein, particularly Example 35, wherein each of the slots of the gearbox housing includes a pair of opposed stop surfaces spaced along the longitudinal axis and positioned to selectively engage and stop movement of the slider arms in predetermined directions.

Example 37: The delivery apparatus of any example herein, particularly any one of Examples 33-36, wherein the second pull body portion includes a pair of guide beams projecting in a direction along the third axial axis and spaced from each other in a direction transverse to the third axial axis.

Example 38: The delivery apparatus of any example herein, particularly Example 37, wherein the pair of guide beams are oriented orthogonally to the pair of slider arms.

Example 39: The delivery apparatus of any example herein, particularly any one of Examples 37-38, wherein an end portion of the gearbox housing includes a pair of channels extending in a direction along the longitudinal axis, and that the pair of guide beams extend into the pair of channels and are movable along the pair of channels in response to movement of the pull body along the longitudinal axis.

Example 40: The delivery apparatus of any example herein, particularly any one of Examples 19-39, wherein the pull body includes a first opening extending in a direction along the third axial axis, and that the gearbox housing includes a second opening extending in a direction along the longitudinal axis, and further includes a guide rod extending through the first and second openings.

Example 41: A delivery assembly comprises a prosthetic heart valve having a frame and at least one actuator coupled to the frame and operable to move the frame between a radially expanded configuration and a radially compressed configuration; a handle comprising a handle body having a cavity and defining a longitudinal axis; an actuator driver configured to releasably engage the at least one actuator; a sleeve member operatively coupled to the actuator driver and movable between a first position in which the actuator driver is retained in engagement with the at least one actuator and a second position in which the actuator driver is released from engagement from the at least one actuator; a pull body disposed within the cavity and coupled to an end portion of the sleeve member, the pull body movable along the longitudinal axis such that movement of the pull body axially displaces the sleeve member between the first position and the second position; and a knob rotatably mounted on the handle body and operatively coupled to the pull body such that rotation of the knob relative to the handle body moves the pull body along the longitudinal axis.

Example 42: The delivery assembly of any example herein, particularly Example 41, wherein the prosthetic heart valve further comprises a valvular structure disposed within and coupled to the frame.

Example 43: The delivery assembly of any example herein, particularly any one of Examples 41-42, wherein the pull body defines an axial axis that is oriented parallel to the longitudinal axis, and that the pull body comprises at least one pin member disposed radially to the axial axis and protruding from a periphery of the pull body.

Example 44: The delivery assembly of any example herein, particularly Example 43, wherein the knob includes a cam slot, and that the knob is disposed around the periphery of the pull body such that the pin member extends into the cam slot.

Example 45: The delivery assembly of any example herein, particularly Example 44, wherein the knob includes an inner surface forming a cylindrical lumen, that the cylindrical lumen defines a central axis that is oriented parallel to the longitudinal axis, and that the cam slot comprises an angled slot portion formed in the inner surface and extending along a path that is angled relative to the central axis.

Example 46: The delivery assembly of any example herein, particularly Example 45, wherein the path is configured such that the pull body moves in a direction parallel to the central axis as the pin member moves along the slot portion.

Example 47: The delivery assembly of any example herein, particularly any one of Examples 41-46, further comprising a gearbox disposed within the cavity and coupled to the actuator driver for rotation of the actuator driver.

Example 48: The delivery assembly of any example herein, particularly Example 47, wherein the pull body is movably coupled to the gearbox.

Example 49: A method of implanting a prosthetic heart valve comprises engaging an end portion of an actuator driver coupled to a handle with an end portion of an actuator coupled to a frame of the prosthetic heart valve; moving a pull body along a longitudinal axis of the handle in a first direction to extend an end portion of a sleeve member coupled to the pull body over the engaged end portions of the actuator driver and actuator; delivering the prosthetic heart valve to an implantation location; radially expanding the prosthetic heart valve to a functional size; moving the pull body along the longitudinal axis of the handle in a second direction that is opposite to the first direction to retract the end portion of the sleeve member from the engaged end portions of the actuator driver and actuator; and releasing the end portion of the actuator driver from the end portion of the actuator.

Example 50: The method according to any example herein, particularly Example 49, wherein each of the moving the pull body along the longitudinal axis of the handle includes rotating a knob operatively coupled to the pull body.

The subject matter has been described with a selection of implementations and examples, but these preferred implementations and examples are not to be taken as limiting the scope of the subject matter since many other implementations and examples are possible that fall within the scope of the subject matter. The scope of the claimed subject matter is defined by the claims.

1. A delivery apparatus for a prosthetic heart valve, the delivery apparatus comprising:
a handle body having a first end, a second end, a cavity between the first end and the second end, and a longitudinal axis extending through the first end, the cavity, and the second end;
an actuator driver having a first driver end portion disposed within the cavity, a second driver end portion disposed outside the cavity, and a first axial axis extending through the first driver end portion and the second driver end portion;
a sleeve member operatively coupled to the actuator driver, the sleeve member having a first sleeve end portion disposed within the cavity, a second sleeve end portion disposed outside the cavity, and a second axial axis extending through the first sleeve end portion and the second sleeve end portion, wherein the second axial axis is parallel to the first axial axis; and
a pull body disposed within the cavity and coupled to the first sleeve end portion, the pull body movable along the longitudinal axis such that movement of the pull body axially displaces the sleeve member relative to the handle body.

2. The delivery apparatus of claim 1, wherein the second driver end portion comprises an engagement member to releasably couple the actuator driver to an actuator of the prosthetic heart valve, and wherein the sleeve member is extendible over the engagement member and retractable from the engagement member by movement of the pull body along the longitudinal axis.

3. The delivery apparatus of claim 1, further comprising a gearbox disposed within the cavity and coupled to the actuator driver for rotation of the actuator driver, wherein the gearbox comprises a gearbox housing enclosing a gear train, and wherein the pull body is movably coupled to the gearbox housing.

4. The delivery apparatus of claim 3, further comprising a knob coupled to the pull body, wherein rotation of the knob relative to the handle body moves the pull body along the longitudinal axis.

5. The delivery apparatus of claim 4, wherein the pull body comprises a first pull body portion, a second pull body portion, and a third axial axis extending through a center of the first pull body portion and a center of the second pull body portion, wherein the third axial axis is parallel to the longitudinal axis.

6. The delivery apparatus of claim 5, wherein the first pull body portion comprises a pin member disposed radially to the third axial axis and positioned on a periphery of the pull body, wherein the knob comprises a cam slot, and wherein the knob is disposed around the pull body such that the pin member extends into the cam slot.

7. The delivery apparatus of claim 6, wherein the knob comprises an inner surface forming a cylindrical lumen, wherein the cylindrical lumen defines a fourth axial axis that is oriented parallel to the longitudinal axis, and wherein the cam slot comprises an angled slot portion formed in the inner surface and extending along a path that is angled relative to the fourth axial axis.

8. The delivery apparatus of claim 6, wherein the first pull body portion comprises a central hub and a radial arm projecting from the central hub, wherein the pin member is formed at an end of the radial arm.

9. The delivery apparatus of claim 6, wherein the second pull body portion comprises a socket extending in a direction parallel to the third axial axis, and wherein the first pull body portion comprises an opening forming an extension of the socket.

10. The delivery apparatus of claim 9, further comprising an actuation tube extending through the opening and socket, wherein the sleeve member is rigidly coupled to the actuation tube, and wherein the actuation tube is rotationally fixed relative to the pull body.

11. The delivery apparatus of claim 5, wherein the first pull body comprises a plurality of pin members disposed radially to the third axial axis and positioned on a periphery of the pull body, and wherein the pin members are angularly offset from each other.

12. The delivery apparatus of claim 11, wherein the knob comprises a plurality of cam slots, and wherein the knob is disposed around the pull body such that each of the pin members is received in one of the cam slots.

13. The delivery apparatus of claim 12, wherein the knob comprises an inner surface forming a cylindrical lumen, wherein the cylindrical lumen defines a fourth axial axis, and wherein each of the cam slots comprises an angled slot portion formed in the inner surface and extending along a path that is angled relative to the fourth axial axis.

14. The delivery apparatus of claim 11, wherein the first pull body portion comprises a central hub and a plurality of radial arms projecting from the central hub and angularly offset from each other, and wherein each of the pin members is formed at an end of one of the radial arms.

15. The delivery apparatus of claim 11, further comprising additional actuator drivers and additional sleeve members, each additional sleeve member operatively coupled to one of the additional actuator drivers.

16. The delivery apparatus of claim 15, wherein the second pull body portion comprises a plurality of sockets extending in a direction parallel to the third axial axis, wherein the first pull body portion comprises a plurality of openings, each of the openings forming an extension of one of the sockets.

17. The delivery apparatus of claim 16, further comprising a plurality of actuation tubes, each of the actuation tubes extending through one of the sockets and the corresponding opening, wherein each of the sleeve member and additional sleeve members is rigidly coupled to one of the actuation tubes, and wherein each of the actuation tubes is rotationally fixed relative to the pull body.

18. The delivery apparatus of claim 7, wherein the second pull body portion comprises a pair of slider arms projecting in a direction parallel to the third axial axis and spaced from each other in a direction transverse to the third axial axis.

19. The delivery apparatus of claim 18, wherein each slider arm comprises an end portion having a pair of opposed surfaces oriented transversely to the third axial axis;
wherein an end portion of the gearbox housing comprises a pair of slots extending in a direction along the longitudinal axis, wherein the pair of slider arms extend into the pair of slots and are movable along the pair of slots in response to movement of the pull body along the longitudinal axis; and
wherein each of the slots of the gearbox housing comprises a pair of opposed stop surfaces spaced along the longitudinal axis and positioned to selectively engage and stop movement of the slider arms in predetermined directions.

20. A method of implanting a prosthetic heart valve, comprising:
engaging an end portion of an actuator driver coupled to a handle with an end portion of an actuator coupled to a frame of the prosthetic heart valve;
moving a pull body along a longitudinal axis of the handle in a first direction to extend an end portion of a sleeve member coupled to the pull body over the engaged end portions of the actuator driver and actuator;
delivering the prosthetic heart valve to an implantation location;
radially expanding the prosthetic heart valve to a functional size;
moving the pull body along the longitudinal axis of the handle in a second direction that is opposite to the first direction to retract the end portion of the sleeve member from the engaged end portions of the actuator driver and actuator; and
releasing the end portion of the actuator driver from the end portion of the actuator.
